(19) United States
(12) Patent Application Publication
(10) Pub. No.: US 2015/0225739 A1
(43) Pub. Date: Aug. 13, 2015

(54) EXPRESSION VECTORS COMPRISING IRES ELEMENT AND THE MULTIPLE EXPRESSION GENE SYSTEM THEREOF

(71) Applicant: National Taiwan University, Taipei City (TW)

(72) Inventors: GUANG-HSIUNG KOU, Taipei City (TW); CHU-FANG LO, Taipei City (TW); SHIH-TING KANG, Taipei City (TW)

(21) Appl. No.: 14/315,793

(22) Filed: Jun. 26, 2014

(30) Foreign Application Priority Data
Feb. 13, 2014 (TW) 103104691

(51) Int. Cl. C12N 15/85

(52) U.S. Cl. CPC C12N 15/85; C12N 2840/60

(57) ABSTRACT

The present invention provides an expression vector comprising an internal ribosome entry site (IRES) element, comprising a sequence of SEQ ID NO: 1, wherein the sequence is an IRES element from a gene icp35 of White spot syndrome virus. The expression vector can be easily operated in insect cells or Crustacean cells and has excellent expression efficiency due to having such an IRES element. A multiple expression gene system is also provided herein, which comprises the expression vector. The system comprising the IRES element can be functioned via transfection, such that the experimental process can be considerably shortened, and thus studying costs will be reduced effectively.

### TECHNICAL FIELD

The present invention is related to an expression vector comprising an internal ribosome entry site (IRES) element and the multigene expression system thereof, and more particularly, to those comprising an IRES element of a gene icp35 in White spot syndrome virus.

### BACKGROUND

In eukaryotes and eukaryotic viruses, the regulation of protein synthesis is either cap-dependent or else uses a cap-independent translation mechanism. In cap-dependent translation, 5′-terminal m7GpppG cap needs to be first recognized by eIF4F, and then combines with a 43S pre-initiation complex for the following reactions such as translation initiation, elongation and termination. While cap-independent translation can initiate the translation within mRNA without any cap structure. Such special translation mechanism includes: internal ribosome entry site (IRES), leaky scanning or translation reinitiation, or the like.

IRES elements, one type of cap-independent translation, are located in the upstream of initiation codon ATG or nearby. Without the help of 5′-terminal cap of mRNA or some translation-related factors, this IRES elements can attract and combine with 40S ribosome subunit directly by its RNA sequence to initiate the translation and synthesize proteins. IRES element consists of RNA sequences that often show stable secondary and/or tertiary structure. Due to the direct attraction and combination with host translation systems by the structure itself, IRES elements can synthesize proteins within mRNA. Recent studies related to IRES translation mechanism (see Fitzgerald, K. D. and B. L. Semler, Bridging IRES elements in mRNAs to the eukaryotic translation apparatus. Biochimica Et Biophysica Acta-Gene Regulatory Mechanisms, 2009. 1789(9-10): p. 518-528.) found that many viruses use IRES translation to regulate viral protein synthesis, which RNA virus studies are in the majority. For example, some viruses in at least three RNA virus classes including: Picornaviruses, Flaviviruses and Dicistroviruses have IRES elements. However, only a very limited number of IRES elements have been reported in DNA viruses, including: Kaposi's sarcoma-associated herpesvirus (KSHV), Epstein-Barr virus (EBV), Herpes simplex virus (HSV), Murine gammaherpesvirus 68 (MHV-68), Marek's disease virus (MDV) and Simian vacuolating virus 40 (SV40).

In addition, previous studies also have shown that White spot syndrome virus (WSSV), a large dsDNA virus, has two IRES elements which are respectively located in the 5′ UTR of vp28 and the coding region of vp31/vp39b to regulate the expression of VP28 and VP39B. In Northern blotting, a wssv019 riboprobe was used to detect two transcripts: a large 5.5-kb polycistronic transcript encoding wssv023, wssv019 and other genes, and a small 1.3-kb monocistronic mRNA encoding only wssv019. (see Chen, L. L., et al., Natural and experimental infection of white spot syndrome virus (WSSV) in benthic larvae of mud crab Scylla serrata. Dis Aquat Org, 2000. 40(2): p. 157-61.) This suggests that the expression of wssv019 might be regulated by a cap-independent mechanism for protein synthesis.

On the other hand, multigene expression systems are mostly applied in research fields such as gene therapy, cancer treatment, vaccine production, or co-expression of different subunit proteins to form protein complexes, etc. Therefore, if a successful development for the multigene expression system which can easily be operated, obtain study results for a short time, and can be widely applied in various cell lines and in vivo multigene expression vectors is made, use value and market potential for the multigene expression system can thus increase significantly. In eukaryotes, co-expression strategies for expressing two or more proteins at the same time can be roughly divided into the following types, including: using two promoters, bidirectional promoter, fusion protein, IRES and 2A-peptides, or the like.

As for dual-promoter, two promoters were constructed in the same vectors and transferred into cells via transfection, but its drawback is that two promoters would mutually interfere and compete transcription materials such as transcription factors and RNA polymerases, leading to the decrease or inactivation in one of both. These results caused inconsistent expression levels between two expressed proteins and failed to achieve the expected effect. As for the use of fusion proteins, it was in doubt about whether proteins could fold accurately and whether their activities could be affected.

Previous published viral IRES elements had a length of about hundreds of nucleotides, such that multigene expression vectors had a limited cloning capacity when constructed. Also, most viral IRES had a lower activity as compared with cap-dependent translation mechanism, and thus caused inconsistent protein expression levels of co-expression. This drawback allowed rare IRES elements to be practically applied in multigene expression systems, such as IRES of Encephalomyocarditis Virus (EMCV) or hepatitis C virus (HCV), which was widely applied in mammalian cells now.

In previous studies, multigene expression for insect cells mostly used above-mentioned co-expression strategies and baculovirus expression systems. Although baculovirus expression systems had a high expression efficiency, it consumed longer time during screening and virus titer evaluation, which took about 1 to 2 weeks to screen recombinant baculoviruses having high vieulence for further researches. Thus, the development for new multigene expression vectors is still needed to accelerate the research progress.

### SUMMARY

In the light of these defects in prior art, one object of the present invention is to provide an expression vector comprising a novel internal ribosome entry site (IRES) element, which can be easily operated in insect cells, shrimp cells or Crustacean cells and has an excellent expression efficiency.

Another object provided herein is a multiple expression gene system, which can shorten the experimental process and reduce studying costs effectively as compared with the conventional baculovirus expression systems.

Thus, the present invention provides an expression vector comprising an internal ribosome entry site (IRES) element, which comprises a sequence of SEQ ID NO: 1.

In some embodiments, the sequence can be an IRES element from a gene icp35 of White spot syndrome virus (WSSV).

In some embodiments, the expression vector can be a dual-gene expression vector.

In some embodiments, the dual-gene expression vector can be a dual-luciferase reporter vector. For example, in a specific embodiment, the dual-luciferase reporter vector can comprise a sequence of SEQ ID NO: 2, but not limited to this.

In other embodiments, the dual-gene expression vector can be a dual-fluorescence reporter vector. For example, in a specific embodiment, the dual-fluorescence reporter vector can comprise a sequence of SEQ ID NO: 3, but not limited to this.

A multigene expression system is also provided, which can comprise any expression vector according to the above-mentioned embodiments.

In some embodiments, the multigene expression system refers to an insect cell expression system or a Crustacean cell expression system. For example, in a specific embodiment, the multigene expression system can be a Spodoptera frugiperda (Sf9) expression system.

Embodiments according to the inventive concept of the present invention are provided such that those skilled in the art can more completely understand the present invention. It should also be understood that the following embodiments are not limited by any of the details of the foregoing description, unless otherwise specified, but rather should be construed broadly within its spirit and scope as defined in the appended claims.

### BRIEF DESCRIPTION OF THE DRAWING

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 shows the results of one example that the highly expressed protein ICP35 was a non-structural protein, wherein:

A shows Western blot analysis results for detected positive controls of purified total WSSV virions: the envelope protein VP28 and the nucleocapsid protein VP51C.

B shows the results for time course analysis of WSSV ICP35 (WSSV019) expression.

FIG. 2 shows the results of one example for dsRNA silencing of icp35, wherein:

A shows RT-PCR analysis results for each experimental group at each time point, wherein: the expression levels of icp35 and ie1 in pleopod was detected at 24 and 48 hpi, and in stomach at 96 hpi were monitored. EF-1α was used as an internal control.

B shows results of cumulative mortalities which were observed and recorded every 12 h.

C shows results of cumulative mortality for the mock-infected PBS controls.

D shows results for time course study of WSSV loads after dsRNA silencing. WSSV copy number and shrimp genomic DNA were measured using the IQ REAL WSSV quantitative system (GeneReach Biotechnology Corp.). (**P<0.005 by one-way analysis of variance [ANOVA]).

FIG. 3 shows the 5′ UTR of WSSV icp35 mRNA containing a functional IRES element in one example, wherein:

A shows schematic representation of the WSSV gene clusters wssv023/wssv019 (icp35), vp31/vp39b/vp11, and vp60b/wssv478/wssv479/vp28. The 5′ UTR fragment of icp35(-468/-1) or the IRES elements of VP39B or VP28 were inserted into the intercistronic region of the bicistronic reporter plasmid ie1/pRL-FL;

B shows results for the ratio of firefly to Renilla luciferase. Bicistronic plasmids of icp35(-468/-1) or IRES₁₂₃₋₉₁₉ or vp28-IRES were transfected into Sf9 cells, and Renilla and firefly luciferase activities were measured at 48 h post-transfection. The ratio of firefly to Renilla luciferase was used as an indicator of IRES activity. The FL/RL ratio for icp35(-468/-1) was set to 100%. Three independent transfection assays were performed, and the mean±SD was calculated.

FIG. 4 shows results of one example for 5′- and 3′-end deletion analysis of the icp35 IRES element, wherein:

A shows RNA MFold prediction of the secondary structure of icp35 5′ UTR at 25° C.;

B shows schematic diagram of the bicistronic vector pRL-FL with the wssv ie1 promoter and the 5′ UTR structure of icp35. Various 5′- and 3′-end deleted fragments in the 5′ UTR of icp35 were subcloned into the intercistronic region between the Renilla luciferase and firefly luciferase;

C shows that Sf9 cells were transiently transfected with the bicistronic plasmids, and at 48 h post-transfection Renilla and firefly luciferase activities were measured. The ratios of firefly to Renilla luciferase for each bicistronic plasmid are shown, and the highest IRES activity [icp35(-198/-1)] was set to 100%. The actual ratio was 1.32±0.20. Three independent transfection assays were performed and the mean±SD was calculated (*P<0.05, ****P<0.00005 by one-way analysis of variance [ANOVA]);

D and FIG. 4E are analogous to the schematics and results in FIG. 4B and FIG. 4C except that the deletion fragments were derived from icp35(-198/1-) instead of the entire icp35 5′ UTR.

FIG. 5 shows results of one example for the icp35(-198/-1) bicistronic plasmid proving that it does not show cryptic promoter activity, wherein:

A shows schematics of bicistronic plasmids containing icp35(-291/-1) and icp35(-198/-1) fragments with the WSSV ie1 promoter (+P) or without the WSSV ie1 promoter (−P);

B shows relative luciferase activities of firefly (shown as gray bar) and Renilla (shown as dark gray bar). Plasmids were transfected into Sf9 cells, and 48 h later FL (gray bar) and RL (dark gray bar) activities were measured. FL and RL data for the control plasmid icp35(-291/-1)(+P) were set to 100%. Three independent transfection assays were performed and the mean±SD was calculated (****P<0.00005, Student's t-test).

FIG. 6 shows results of one example for the ie1/pRL-icp35(-198/-1)-FL bicistronic transcript which was proven to have no internal splice sites, wherein:

A shows schematic diagram of the ie1/pRL-FL-based bicistronic construct and the two primers used for reverse transcription (RT)-PCR. The primer set P1/P2 represented ie1P(+3/+31)-F/pRL-FL-R1241 (see Table 1);

B shows RT-PCR results for total RNA extracted from Sf9 cells transfected (T) or untransfected (UT) with the ie1/pRL-icp35(-198/-1)-FL bicistronic plasmid. In the negative RT(−) controls, RTase was omitted to verify that the RT-PCR products were specifically amplified from RNA and not from any contaminating plasmid DNA. The DNA lane denoted the PCR product from the ie1/pRL-icp35(-198/-1)-FL plasmid and was used as a positive size control. Lane M shows 1 kb DNA ladder I markers (LAMDA Biotech Inc.), and lane N was the negative control (no cDNA);

C shows results for mRNA expression levels of Renilla luciferase (RL) and firefly luciferase, which was measured at 48h post-transfection, and total RNA was isolated from ie1/pRL-FL-transfected Sf9 cells and from Sf9 cells transfected with the ie1/pRL-icp35(-198/-1)-FL bicistronic plasmid. Quantitative RT-PCR was performed to analyze the mRNA expression levels of Renilla luciferase (RL) and firefly luciferase was calculated as 2^{-[Ct(FL)-Ct(RL)]} and the ratio of firefly luciferase to Renilla luciferase of ie1/pRL-FL was set to 1. The data represented the mean±SD from three independent transfection experiments.

FIG. 7 shows results of one example for Northern blot analysis of the RNA transcripts derived from icp35 IRES-based bicistronic baculoviruses detected only a single band of bicistronic mRNA of the expected size. Total RNA (30 μg) extracted from AcMNPV-RL/icp35 IRES/FL baculovirus-infected (lane 1) or uninfected (lane 2) Sf9 cells which were electrophoresed, transferred to nylon membrane, and then hybridized with DIG-labeled firefly luciferase probes, wherein:

A shows results of Northern blot analysis, wherein the arrow indicated the bicistronic RNA transcript and the open arrow indicated the position of the 18s rRNA;

FIG. 7B shows results for Methylene blue staining of the 18S rRNA that was used as a loading control

FIG. 8 shows results of one example indicating that the FL activity was not a result of ribosomal read-through.

A shows schematic diagrams of bicistronic plasmids containing icp35(-198/-1) with or without a stable stem-loop (SL) upstream of the RL ORF;

FIG. 8B shows relative luciferase activities of firefly (FL) and Renilla (RL), which was measured 48 h later by transient transfection of Sf9 cells with each plasmid. Fold changes are shown with respect to the corresponding control levels for ie1/pRL-icp35(-198/-1)-FL, which were set to 100%. Three independent transfection assays were performed and the mean±SD was calculated (***P<0.0005, Student's t-test).

FIG. 9 shows results of one example for the reduced IRES activity of icp35(-198/-1) when eFI2α was inactivated by tunicamycin-induced ER stress, wherein:

A shows schematic diagram of the bicistronic plasmid containing the icp35(-198/-1) IRES element;

B shows relative luciferase activities of firefly (FL) and Renilla (RL). The bicistronic plasmid ie1/pRL-icp35(-198/-1)-FL was transiently transfected to Sf9 cells, and 24 h later, the cells were treated with tunicamycin (2.5 μg/ml) or DMSO (100%). Luciferase activities were measured at 15 h post-transfection. Luciferase activities are expressed relative to the levels in untreated (control) cells, which were set to 100%.

C shows results for Western blot analysis in which the level of phosphorylated eIF2α in the Sf9 cells at 15 h post-transfection was detected by Western blot analysis using the antibodies for phospho-eIF2α and total eIF2α. (*P<0.05, ***P<0.0005 by one-way analysis of variance [ANOVA]).

FIG. 10 shows results of one example for icp35 IRES-mediated translation affected by quinacrine (QC), wherein:

A shows schematic diagram of the IRES-containing bicistronic reporter plasmid;

B shows relative luciferase activities of firefly (FL) and Renilla (RL). Bicistronic plasmids were transfected into Sf9 cells with or without QC. Luciferase activities were measured at 48 hour post-transfection. The Figure shows changes relative to the corresponding untreated mock controls (0 μM), which was set to 100%. Three independent transfection assays were performed and the mean±SD was calculated (*P<0.05, **P<0.005 by one-way analysis of variance [ANOVA]);

C shows cumulative mortality of shrimp. Shrimp (L. vannamei; 4 g mean weight; 14 shrimp per group) were first injected with 50 n1 WSSV inoculum or PBS, and then immediately injected with QC (5 μg/g) or PBS. The cumulative mortality of each group was recorded every 12 h, with dead shrimp being removed from the tank as soon as possible. Data were analyzed using a Kaplan-Meier log rank×2 test (Graphpad). Asterisks indicated significant cumulative differences between groups (*P<0.05, ***P<0.0005);

D shows the WSSV infection level in the WSSV-challenged groups. The swimming legs from some of the dead shrimp in the WSSV-challenged groups were tested with an IQ2000 kit to determine the WSSV infection level. The hpi above each lane indicated time of collection. The bands of size 296, 550, and 910 bp represen WSSV genes, and the band of size 848 bp represents shrimp genomic DNA. Samples for the marker bands (M) of size 333, 666 and 840 bp, and the positive infection controls (20, 200, and 2000 copies/reaction; right hand panel) were provided by the kit. The positive infection controls respectively represent a light (+), moderate (++), and heavy (+++) infection. Lane N is the negative control;

E shows the WSSV infection level in two other replicate groups (WSSV+PBS and WSSV+QC). Two other replicate groups (WSSV+PBS and WSSV+QC) were used to collect live shrimp samples at 1, 2, 6 and 9 days post-injection (dpi). WSSV infection levels were determined using the IQ2000 kit as described above.

FIG. 11 shows results of one example for icp35 IRES-mediated translation which was preferentially inhibited by RPS10 knockdown, wherein:

A shows the expression levels of RPS10, RPS19 in Sf9 cells at 48h post-transfection which were monitored by RT-PCR analysis. EF-1α was used as an internal control;

B shows relative luciferase activities of firefly (FL) and Renilla (RL). Sf9 cells were co-transfected with 0.3 μg of bicistronic vector plus 0.1 μg dsRNA using Effectene (Qiagen) according to the manufacturer's protocol. At 72 h post-transfection the cells were assayed using dual luciferase reagents (Promega) following the manufacturer's protocol. (***P<0.0005 by one-way analysis of variance [ANOVA] with three replicates.)

FIG. 12 showed the result for the expression of a bicistronic plasmid containing the icp35 IRES element, ie1/pIZ-V5-DR-icp35 IRES-EGFP, which was transfected into Sf9 cells using Effectene transfection reagent (Qiagen). An empty vector without any IRES element, ie1/pIZ-V5-DR-EGFP, was used as a negative control.

### DETAILED DESCRIPTION

Preferred embodiments of the present invention will be described below in more detail with reference to the accompanying drawings. The present invention may, however, be embodied in different forms and should not be constructed as limited to the embodiments set forth herein. Other objectives, advantages, and novel features of the invention will become more apparent from the following detailed description when taken in conjunction with the accompanying drawings.

First, one embodiments of the present invention provided an expression vector comprising an internal ribosome entry site (IRES) element having a sequence of SEQ ID NO: 1. The IRES element was from a gene icp35 of White spot syndrome virus (WSSV) encoding a highly expressed non-structural protein, which could be transcripted to a polycistronic mRNA with other genes. The expression vector was designed respectively to form a dual-luciferase reporter vector (ie1/pRL-icp35 IRES-FL, including a sequence of SEQ ID NO: 2) and a dual-fluorescence reporter vector (ie1/pIZV5-DR-icp35 IRES-GFP, including a sequence of SEQ ID NO: 3) for the following examples.

A. Preparation and Analysis for the Dual-Luciferase Reporter Vector (Ie1/Pr1-Icp 35 Ires-F1, Including a Sequence of SEQ ID NO: 2)

Materials

Experimental Animals

The Pacific white shrimp Litopenaeus vannamei used in these examples were all WSSV-free, as confirmed by using an IQ2000TM WSSV diagnostic kit (GeneReach Biotechnology Corp.). The shrimp (mean weight 4 g) were obtained from a culturefarm in Tung Kang, Taiwan, or from the Aquatic Animal Center in National Taiwan Ocean University, and were acclimatized in the laboratory in water tanks with a salinity of 30±1 ppt at 26±1° C. for at least 3-5 days before the experiments.

### Example 1

### Preparation Of WSSV Inoculum

The virus used in this example was the WSSV Taiwan isolate WSSV-TW (GenBank accession no. AF440570), which originated from a batch of WSSV-infected Penaeus monodon shrimp collected in Taiwan in 1994. The preparation of the WSSV inoculums was followed the methods described by Tsai et al. (see Tsai, M. F., G. H. Kou, H. C. Liu, K. F. Liu, C. F. Chang, S. E. Peng, H. C. Hsu, C. H. Wang, and C. F. Lo. 1999. Long-term presence of white spot syndrome virus (WSSV) in a cultivated shrimp population without disease outbreaks. Dis Aquat Organ 38:107-114, which was incorporated herein by reference.)

Briefly, a 0.5 g frozen sample of infected P. monodon carapace was ground together with 4.5 ml of 0.9% NaCl until the mixture became homogenized. After centrifugation at 1,000×g for 10 minutes at 4° C., the supernatant was filtered with a 0.45 μm filter (Millipore), and 100 μl of 100× dilution virus was used to infect adult specific-pathogen-free (SPF) Litopenaeus vannamei (mean weight 35 g). Collected hemolymph from moribund shrimp was centrifuged at 1,000×g for 10 minutes at 4° C., and the supernatant was diluted 5× with PBS. This suspension was stored at −80° C. and used as a viral stock.

### Example 2

### Purification Of WSSV Virions

Healthy Procambarus clarkii crayfish were used for this procedure. The WSSV-TW (GeneBank accession no. AF440570) viral stock was diluted with PBS (1:2.5 dilution in PBS; 5 μl/g of body weight) and injected intramuscularly into healthy crayfish. After 4 to 6 days, hemolymph was extracted from moribund crayfish, and virions were purified as described previously. (see Genomic and proteomic analysis of thirty-nine structural proteins of shrimp white spot syndrome virus. Journal of Virology 78:11360-11370, which was incorporated herein by reference.)

### Example 3

### Western Blot Analysis

Shrimp stomachs from healthy (0 hour post-injection; hpi) and WSSV-infected shrimp (at 12, 24, 36, 48, 60, and 72 hpi) were ground in liquid nitrogen, and lysed with ⅓× ice-cold PBS with complete protease inhibitor cocktail (Roche). Supernatants were obtained after 12000×g centrifugation at 4° C. for 15 min, and concentrations of total protein were quantified using the Bio-Rad Bradford Protein Assay. Total protein (18 μg) of each extract was separated on 15% SDS-PAGE, and the gel was transferred onto polyvinyl difluoride (PVDF) membrane (PerkinElmer) using the wet transfer method (Hoefer apparatus).

For the time course expression experiments, the blots were blocked with 5% non-fat milk in TBS buffer for 16 h overnight at 4° C. Next, the blots were probed with primary antiserum against ICP35, VP28, VP51C or β-tubulin diluted 1:10,000 in 5% non-fat milk in TBST (0.05% of Tween 20 in TBS buffer) for 1 h at room temperature (RT), and this was followed by washing three times for 10 min at RT with TB ST. The blots were then probed with anti-Rabbit IgG secondary antibody (Santa Cruz Biotechnology) diluted 1:10,000 in 5% non-fat milk in TBST for 1 h at RT, and washed three times for 10 min at RT with TBST. The blots were incubated with chemiluminescent substrate using Western Lightning® Plus-ECL reagents (PerkinElmer) and exposed to film for signal detection.

### Example 4

### Synthesis Of dsRNA

The genes for wssv icp35, EGFP, Spodoptera frugiperda RPS10, and RPS19 were amplified by the primer sets icp35-F1/icp35-R687, EGFP-F/EGFP-R, Sf-RPS10-F/Sf-RPS10-R, and Sf-RPS19-F/Sf-RPS19-R respectively (Table 1) with the following profile: 94° C. for 3 minutes; 30 cycles at 94° C. for 30 seconds, 55° C. for 30 seconds and 72° C. for 30 seconds (30 cycles), and then a final extension at 72° C. for 20 minutes. A T7 promoter was attached to the 5′-end of these purified PCR products by amplification with, the primer sets icp35-dsRNA-T7-F1/icp35-R687, EGFP-T7-F/EGFP-R, Sf-RPS10-dsRNA-T7-F/Sf-RPS10-121 R and Sf-RPS19-dsRNA-T7-F/Sf-RPS19-R respectively (Table 1). The T7 promoter was likewise attached to the 3′-end with the respective primer sets icp35-F1/icp35-dsRNA-T7-R687, EGFP-F/EGFP-T7-R, Sf-RPS10-F/Sf-RPS10-dsRNA-T7-R, and Sf-RPS19-F/Sf-RPS19-dsRNA-T7-R (Table 1).

1 μg of each PCR product having 5′-end and 3′-end T7 promoter was used as DNA template, and then used to perform in vitro transcription by RiboMAX™ Large Scale RNA Production System-T7 (Promega) according to the manufacturer's instructions. The resulting sense and antisense ssRNAs generated from the two kinds of DNA template were mixed together equally, heated at 70° C. for 10 minutes, and incubated at RT for at least 20 minutes to anneal the two complementary ssRNAs into dsRNA. The DNA templates were removed by adding DNase I (Invitrogen), and phenol/chloroform extraction and ethanol precipitation were used to obtain the dsRNA products. The concentration of the dsRNA products was estimated using a NanoDrop® (ND-1000) spectrophotometer.

### Example 5

### dsRNA-Mediated Interference Assay

For the dsRNAi experiment, shrimps were randomly divided into two sets, and then each set was further divided into three experimental groups. Two experimental groups in one set of 10-12 shrimp were injected with icp35 dsRNA or EGFP dsRNA at a concentration of 4 μg (1 μg/g body weight) in 50 μl of PBS, and another group with 50 μl of PBS only as control. Two days later, these groups were injected with 50 μl of WSSV inoculum (50× dilution of the virus stock), while three experimental groups in another set was injected with 50 μl of PBS as a negative control. At 0, 24, 48, 96 hpi, pleopod samples were excised from 3 randomly selected shrimp in each group. One pleopod from each shrimp was subjected to RT-PCR to evaluate the gene expression level after dsRNAi-mediated gene knockdown, and a second pleopod was subjected to real-time PCR to quantify the WSSV viral load. Both of these procedures are described below. In addition, a second replicate was made of this entire experiment. In this replication, instead of taking samples from the shrimp, the mortality was observed and recorded in each group every 12 h.

### Example 6

### Reverse Transcription-PCR Analysis

Total RNA (1 μg) isolated from the pleopod samples by Trizol reagent (Invitrogen) was pretreated with DNase I (Invitrogen) and then reverse transcribed by SuperScript III Reverse Transcriptase (Invitrogen). RT-PCR was performed with primer sets for icp35, ie1, and EF-1α (Table 1) using the following profile: 94° C. for 3 minutes; 30 cycles at 94° C. for 30 seconds, 55° C. for 30 seconds, and 72° C. for 30 seconds (35 cycle); a final extension at 72° C. for 20 minutes.

### Example 7

### Absolute Quantification of WSSV Loads

The IQ REAL WSSV quantitative system (GeneReach Biotechnology Corp.) was used to absolutely quantify the WSSV loads in the dsRNA-mediated gene silencing experiments. From the pleopod samples taken at 48 and 96 hpi, shrimp genomic DNA was isolated using the silica-based resin supplied with the commercial kit and quantified according to the manufacturer's instructions using a TaqMan assay strategy. Reactions were performed on an ABI PRISM 7300. The WSSV load was calculated by the relative ratio of copy number of WSSV genomic DNA to shrimp genomic DNA. WSSV load data were presented as mean±SD (standard deviation) for 3 shrimp from each group, and one-way analysis of variance (ANOVA) tests were used to check for significant difference with the P value<0.005.

### Example 8

### Plasmid Construction

Schematics for all of the plasmids used in all Examples can be found in the Results section. The backbone plasmid used for the dual luciferase assays was constructed as described previously (see Kang, S. T., J. H. Leu, H. C. Wang, L. L. Chen, G. H. Kou, and C. F. Lo. 2009. Polycistronic mRNAs and internal ribosome entry site elements (IRES) are widely used by white spot syndrome virus (WSSV) structural protein genes. Virology 387:353-363, which was incorporated herein by reference). Basically, the firefly luciferase from pGL3 plasmid (Promega) was inserted into the pRL-null plasmid (Promega) to give the dual luciferase plasmid T7/pRL-FL (see Bieleski, L., and S. J. Talbot. 2001. Kaposi's sarcoma-associated herpesvirus vCyclin open reading frame contains an internal ribosome entry site. J Virol 75:1864-1869, which was incorporated herein by reference), but before transient DNA transfection in Spodoptera frugiperda Sf9 cells, the T7 promoter was replaced by the WSSV ie1 promoter (−94/+52). This substitution was achieved by PCR amplification with the primers ie1-promoter-SacI-F and ie1-promoter-NheI-R (Table 1) to clone the ie1 promoter into the SacI-NheI sites of T7/pRL-FL to produce the construct ie1/pRL-FL. The putative IRES elements in the 5′ UTR of icp35 and its antisense sequence (as a negative control) were PCR amplified using KOD+Taq polymerase (TOYOBO) with the primer sets listed in Table 1 to clone each respective element into the reporter construct ie1/pRL-FL to generate the corresponding plasmids. The two previously published WSSV IRES elements, IRES₁₂₃₋₉₁₉ (i.e. vp39b-IRES) and vp28-IRES, were also cloned into the reporter plasmid and used as controls for comparison. Primer sets were listed in Table 1. The empty vector ie1/pRL-FL was used as a negative control.

For the promoterless assays, the ie1 promoters of ie1/pRL-icp35(-291/-1)-FL and ie1/pRL-icp35(-198/-1)-FL were removed by digestion with SacI and NheI followed by Klenow treatment and religation to generate pRL-icp35(-291/-1)-FL(−P) and pRL-icp35(-198/-1)-FL(−P), respectively.

To rule out the possibility of ribosomal read-through, a 28-bp stable stem-loop structure with a free energy of −62 kcal/mol (5′-GCTAGCGGTACGGCAGTGCCGTACGACGAATTCGT CGTACGGCACTGCCGTACCGCTAGC-3′, SEQ ID NO: 4) was introduced upstream of the Renilla luciferase ORF using the NheI site as described previously (see Bieleski, L., and S. J. Talbot. 2001. Kaposi's sarcoma-associated herpesvirus vCyclin open reading frame contains an internal ribosome entry site. J Virol 75:1864-1869, which was incorporated herein by reference.) to generate the plasmid ie1/SL-pRL-icp35(-198/-1)-FL.

### Example 9

### RNA Secondary Structure Prediction

The RNA secondary structure of the putative IRES element of icp35(-468/-1) was predicted by the RNA Folding Form software on the mfold Web Server (http://mfold.rna.albany.edu/?q=mfold/RNA-Folding-Form2.3). The folding temperature was set to 27 since the optimal temperature for WSSV infection was 27.

### Example 10

### IRES Activity Assay for Transfected Sf9 Cells

For the IRES activity assays, Sf9 cells were seeded in 24-well plates (1.2×10⁵ cells/well) and grown in Sf-900 II medium (Invitrogen) supplemented with 10% Fetal Bovine Serum (FBS; Gibco®) overnight at 27° C. Plasmid DNAs (0.4 μg of plasmid DNA per well) were transfected into the cells using Effectene transfection reagent (Qiagen) according to the manufacturer's recommendations. Cells were harvested 48 h after transfection and analyzed for dual luciferase activities using the Dual-Luciferase® Reporter Assay System (Promega). Briefly, transfected cells were washed twice with 1×PBS, lysed with 100 ul of passive lysis buffer, and then incubated for 15 min at RT on an orbital shaker with gentle shaking. Luciferase activities in the cell lysates (10 μl) were measured with a Labsystems benchtop luminometer. The ratio of firefly luciferase activity to Renilla luciferase activity was used as an index of IRES activity. Transfection assays were performed in triplicate with three independent experiments. Data are presented as mean±SD (standard derivation) from the three independent triplicate experiments.

1. Abnormal Splicing Test

To check whether abnormal splicing occurred during transfection, RT-PCR was performed with the primer set P1/P2 (Table 1) using the following profile: 94° C. for 3 minutes; 30 cycles at 94° C. for 30 seconds, 60° C. for 30 seconds, and 72° C. for 2 minutes (32 cycle or 40 cycle); a final extension at 72° C. for 20 minutes. The PCR products were then cloned into the vector pGEM-T (Promega) and sequenced. In addition, a quantitative real-time PCR was performed using the KAPA SYBR® FAST Universal Kit (Kapa Biosystems) on an ABI Prism 7300 sequence detection system (Applied Biosystems) according to the manufacturer's instructions. The mRNA expression levels of the Renilla luciferase and firefly luciferase genes were detected with the primer sets

Rluc-qPCR-F/Rluc-qPCR-R (Table 1) and Fluc-qPCR-F/Fluc-qPCR-R (Table 1), respectively. The mRNA expression level of Sf9EF-1α was used as an internal control (Table 1).

2. IRES Activity Experiment by the Inducement of Endoplasmic Reticulum (ER) Stress with Tunicamycin

For the tunicamycin experiments, Sf9 cells were transfected with the bicistronic plasmid ie1/pRL-icp35(-198/-1)-FL (1.0 μg of plasmid DNA per well of a 24-well plate) using the SuperFect transfection

reagent (Qiagen) according to the manufacturer's recommendations, and 2.5 μg/ml final concentration of tunicamycin (Sigma) or DMSO control (100%) was added at 24 h post-transfection.

After 15 h of treatment, cells were harvested. One replicate of the harvested cells was subjected to a dual luciferase assay as described above. The other replicate was subjected to Western blotting to monitor the level of the phosphorylated proteins. For this assay, 2× sample buffer was added to the cells and the mixture was boiled for 10 minutes. An aliquot (15 μl) was separated on 15% SDS-PAGE and transferred onto the PVDF membrane as described above. The blots were then blocked with 5% BSA in TBST (0.01% of Tween 20 in TBS buffer) for 1 h at RT, and probed with the commercial primary antibodies Phospho-eIF2α (Ser51) (Cell Signaling) or EIF2S1 (Abeam). The antibodies were diluted 1:1,000 in 2.5% non-fat milk in TBST and applied for 16 h at 4° C. This step was followed by washing three times for 10 min at RT with TBST. The blots were then probed with anti-Rabbit or anti-Mouse IgG secondary antibody (Santa Cruz Biotechnology) diluted 1:10,000 in 2.5% BSA in TBST for 1 hr at RT, and washed three times for 10 min at RT with TBST. Western Lightning® Plus-ECL reagents were used for visualization as described above.

3. Quinacrine (QC) Effect on Transcription Regulated by Icp35 IRES

For the quinacrine (QC) experiment, Sf9 cells were first transfected with the bicistronic plasmid ie1/pRL-icp35(-198/-1)-FL (0.4 μg of plasmid DNA per well of a 24-well plate) using the Effectene transfection reagent (Qiagen) according to the manufacturer's recommendations, and then quinacrine dihydrochioride (Sigma-Aldrich; dissolved to a 10 mM stock solution in ddH₂O, and filtered through a 0.22 μm filter) was added directly to the well at a final concentration of 25 μM or 30 μM. Renilla and firefly luciferase activities were measured after 48 h of treatment.

Transfection assays were performed in triplicate with three independent experiments. Data are presented as mean±SD (standard derivation) from the three independent triplicate experiments.

4. The Effect of RPS10 and RPS19 Genes on IRES Activity by Double-Stranded RNA Interference (dsRNAi)

For the dsRNAi experiment, Sf9 cells were co-transfected with the bicistronic plasmid ie1/pRL-icp35(-198/-1)-FL (0.3 μg of plasmid DNA per well of a 24-well plate) and RPS10 or RPS19 dsRNA (0.1 μg) using the Effectene transfection reagent (Qiagen) according to the manufacturer's recommendations. At 72 h post-transfection, cells were harvested and the dual luciferase activities were measured as described above.

### Example 11

### Generation of an IRES-Based Recombinant Virus

To generate an icp35 IRES-based bicistronic baculovirus transfer vector, the icp35(-198/-1) fragment was amplified with the primer set icp35(-198)-BamHI/icp35(-1)-XhoI (Table 1) and cloned into the pBacPAK9 transfer vector (Clonetech) using the BamHI and XhoI sites. Sf9 cells were co-transfected with the IRES-based transfer vector and a Bsu36 I-digested BacPAK6 viral DNA to produce an AcMNPV-RL/icp35 IRES/FL recombinant virus according to the manufacturer's instructions (Clonetech).

### Example 12

### Northern Blot Analysis

Sf9 cells were infected by AcMNPV-RL/icp35 IRES/FL recombinant viruses for 4-5 days. The cells were then harvested and Trizol reagent (Invitrogen) was used to extract total RNA. The total RNA was electrophoresed on a 1% formaldehyde gel, transferred to a positively charged membrane (Roche Applied Science), and detected by digoxigenin (DIG)-labeled RNA probes for firefly luciferase as described below. For a negative control, the same protocols were applied to uninfected Sf9 cells.

The DIG-labeled RNA probe was created as described previously (see Kang, S. T., J. H. Leu, H. C. Wang, L. L. Chen, G. H. Kou, and C. F. Lo. 2009. Polycistronic mRNAs and internal ribosome entry site elements (IRES) are widely used by white spot syndrome virus (WSSV) structural protein genes. Virology 387:353-363, which was incorporated herein by reference). Briefly, a partial fragment of firefly luciferase was amplified with the primer set FL-F/FL-R (Table 1), ligated with T7 adaptor (Table 1) using T4 ligase (Promega), and then subjected to PCR amplification with a primer set FL-F/T7 adaptor primer 1. The resulting DNA template (200 ng) was then reacted in vitro at 37° C. for 2 h with 5× DIG-RNA labeling mix (Roche Applied Science) and T7 RNA polymerase (Promega). The reaction mixture was then treated with DNase I (RNase-free; Invitrogen) to remove the DNA template and leave only the DIG-labeled RNA probe. For hybridization with the target RNA, and the DIG-labeled RNA probes were denatured by heating at 95° C. for 5 min and cooling on ice for 1 min, and then mixed with preheated (68° C.) DIG Easy Hyb (Roche Applied Science). Hybridization then proceeded for 16 h at 68° C. (DIG System Users Guide from Roche Applied Science).

After hybridization, the membrane was incubated with Anti-Digoxigenin-AP Fab fragment (1:20,000 dilution) (Roche Applied Science), detected with CDP-Star (ready-to-use; Roche Applied Science), and overexposed to X-ray film at RT overnight.

### Example 13

### In Vivo Quinacrine Assay and WSSV Challenge Experiment

To determine the optimal QC dosage for the challenge experiment, four groups of shrimp (20 shrimp per group) were injected with different doses (0.5 μ/g, 2.5 μg/g, or 5 μg/g body weight) of QC (10 mg/mL stock solution in PBS) or with PBS (negative control). The mortality of each group was observed and recorded every 12 h.

All but one of the shrimp (in the 0.5 μg/g group) survived for 2 weeks, and the highest dosage of 5 μg/g was therefore used in the following WSSV challenge experiment. For the WSSV challenge experiment, groups of shrimp (14 shrimp per group) were injected either with WSSV (50 μl of 100× dilution of the virus stock) or with PBS as a negative control. These first injections were then immediately followed by a second injection of 50 μl of either QC (5 μg/g) or PBS. Pleopods were excised from 3 randomly selected shrimp from each group at 0, 1, 2, 6 and 9 days post-injection (dpi) and tested for WSSV infection as described below. A second replicate of this entire experiment was performed at the same time. No pleopods were collected from the shrimp in this second replicate. Instead, mortality was observed and recorded every 12 h.

### Example 14

### Determination of WSSV Infection Status

A commercial WSSV diagnostic kit (IQ2000 WSSV Detection and Prevention System, GeneReach Biotechnology Corp.) was used to determine WSSV infection levels. Briefly, genomic DNA extracted from the shrimp pleopod samples was isolated using the DTAB/CTAB extraction procedure according to the manufacturer's instructions. Next, competitive nested PCR analysis was performed using the extracted DNA samples. The PCR products were analyzed by electrophoresis with a 2% agarose gel. WSSV infection levels were determined according to the pattern of bands indicated in the kit's instructions.

Results

1. WSSV ICP35 was a Highly Expressed Non-Structural Protein

wssv019 was originally thought to encode a structural protein (see Chen, L. L., J. H. Leu, C. J. Huang, C. M. Chou, S. M. Chen, C. H. Wang, C. F. Lo, and G. H. Kou. 2002. Identification of a nucleocapsid protein (VP35) gene of shrimp white spot syndrome virus and characterization of the motif important for targeting VP35 to the nuclei of transfected insect cells. Virology, which was incorporated herein by reference), and to confirm this, Western blot analysis of purified WSSV virions was performed using the anti-WSSV019 antibody. Anti-VP28 (envelope protein)

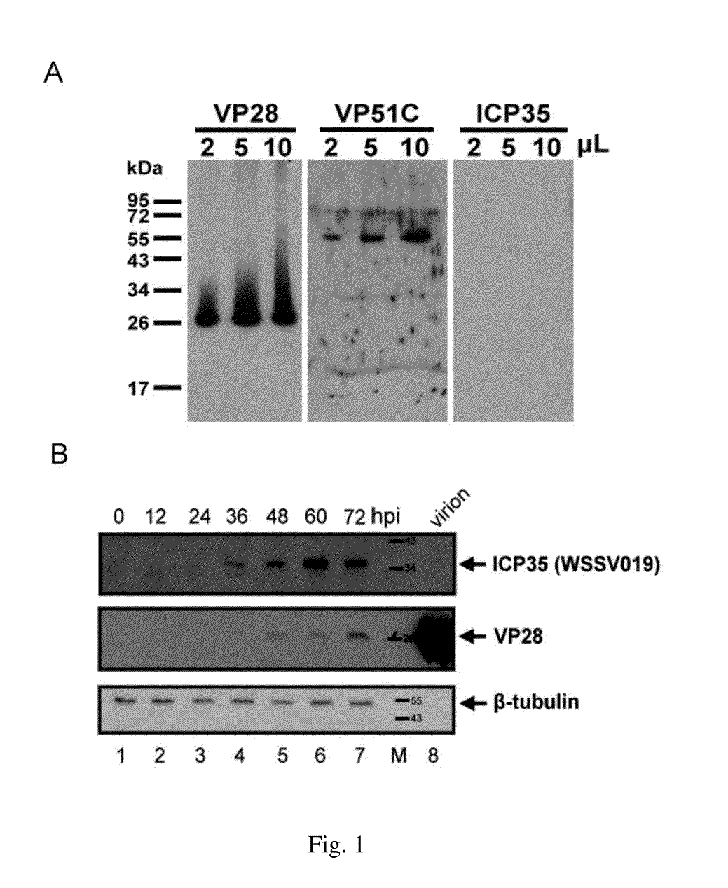

and anti-VP5 1C (nucleocapsid protein) antibodies were used as positive controls. However, since no signal was detected from purified WSSV virions by the anti-WSSV019 antibody (FIG. 1A), we concluded that wssv019 does not in fact encode a structural protein. From now on, we therefore refer to this protein not as VP35 but instead by the new name of ICP35.

A time course study of ICP35 protein levels in shrimp pleopod was performed using Western blot analysis. FIG. 1B shows ICP35 was expressed from 24 to 72 hpi, and reached the highest level at 60 hpi.

2. dsRNA Silencing of Icp35 Reduced WSSV Replication in Shrimp

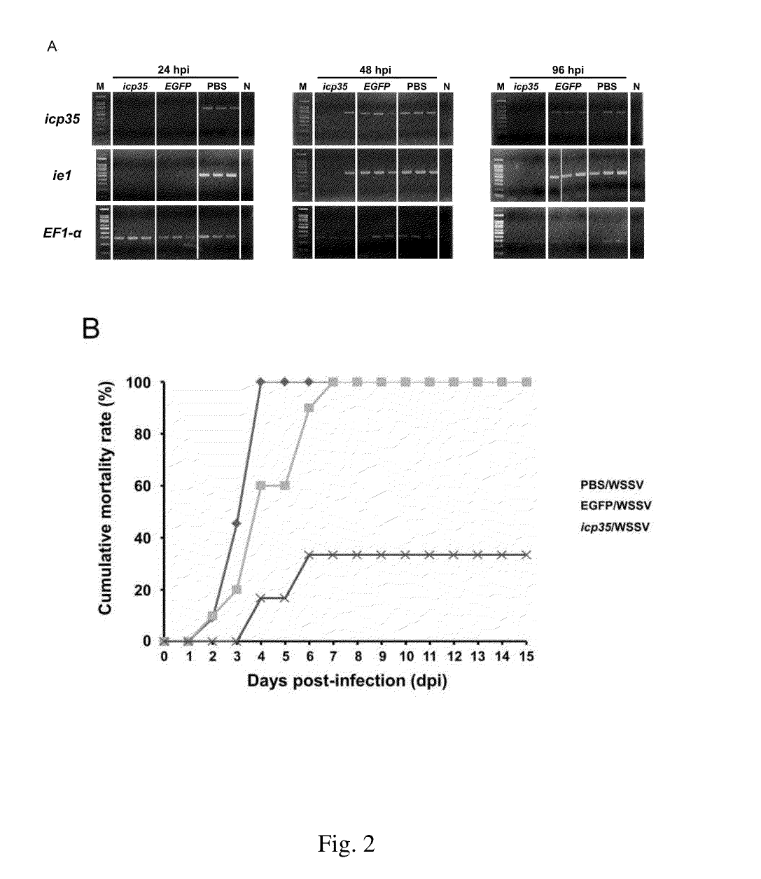

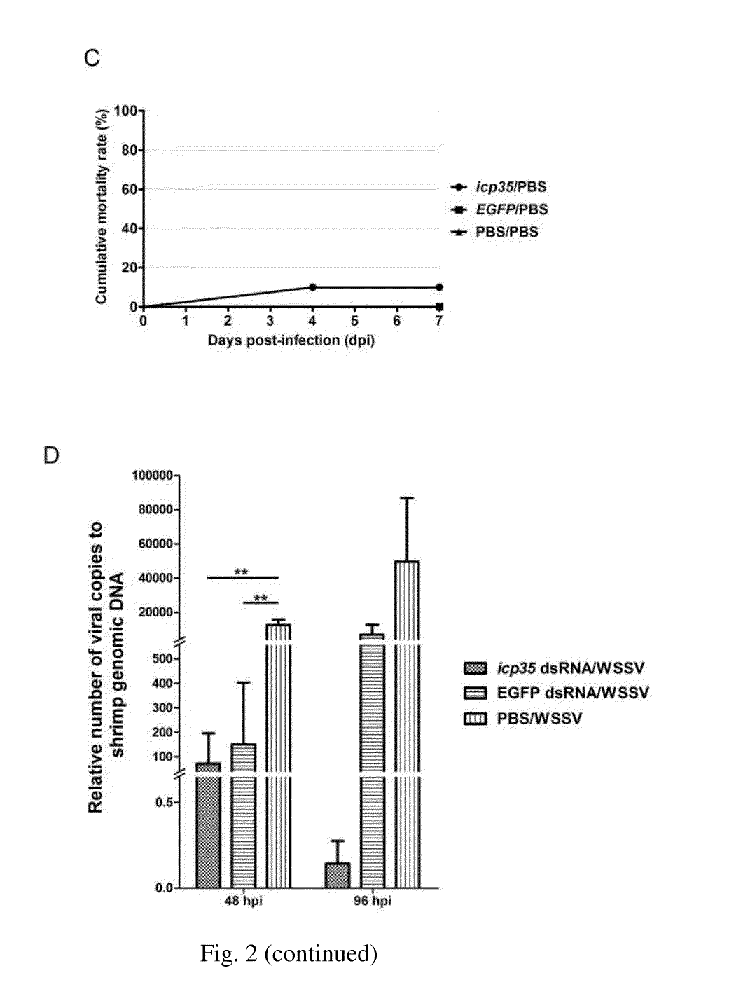

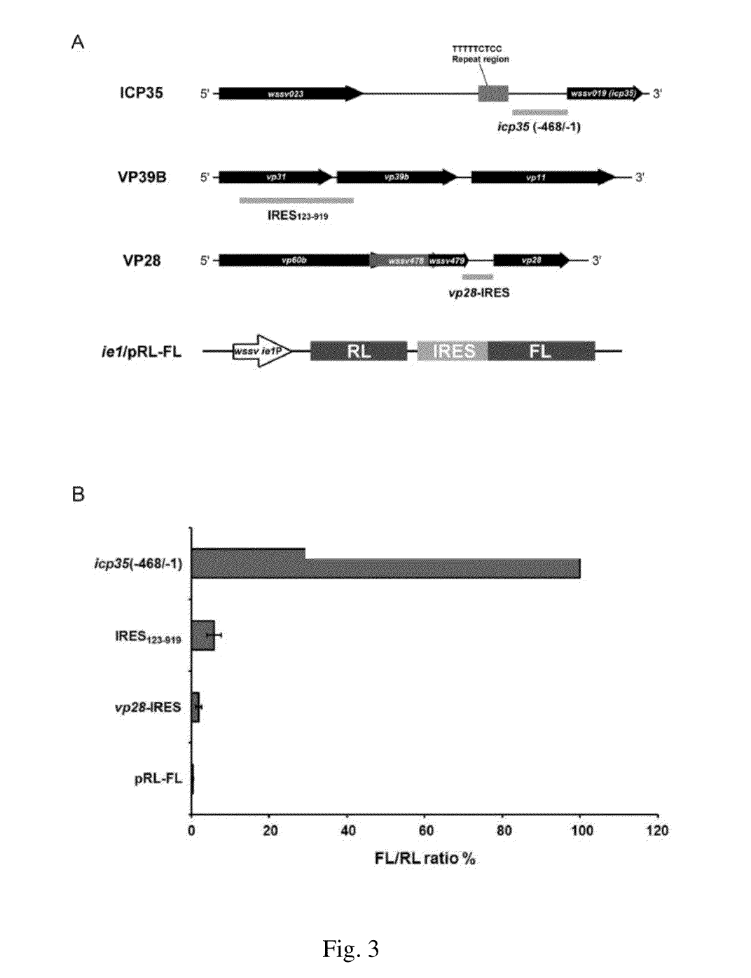

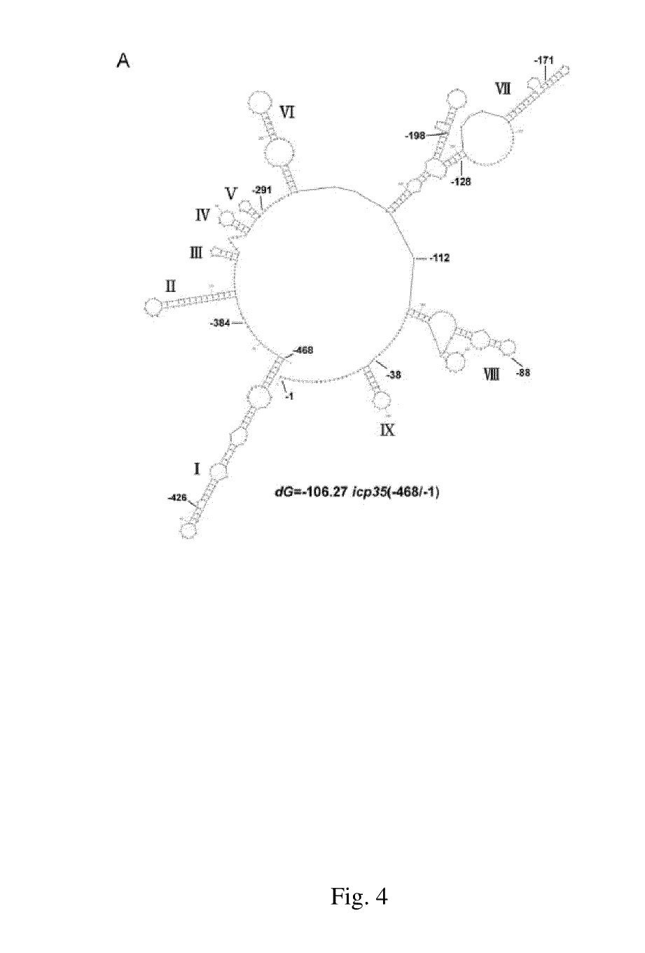

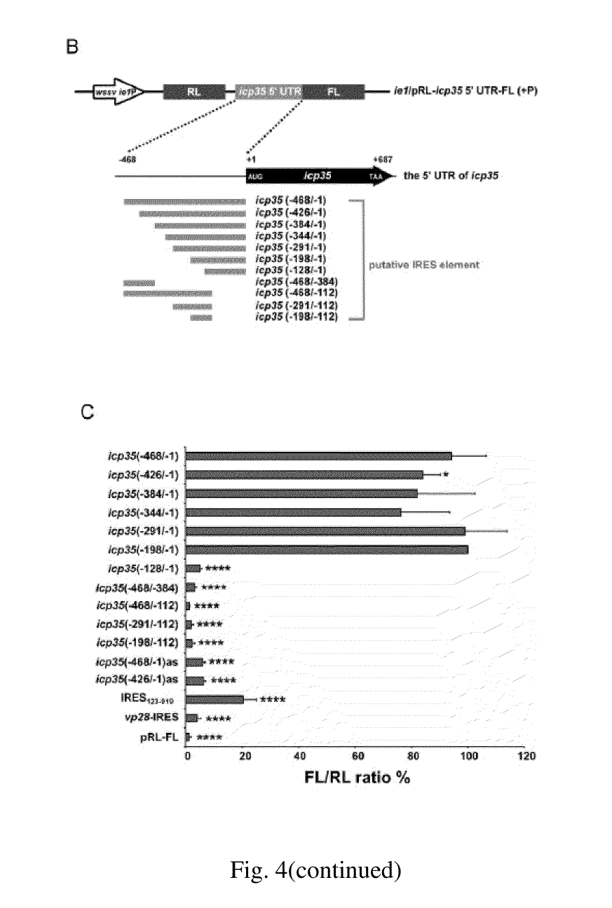

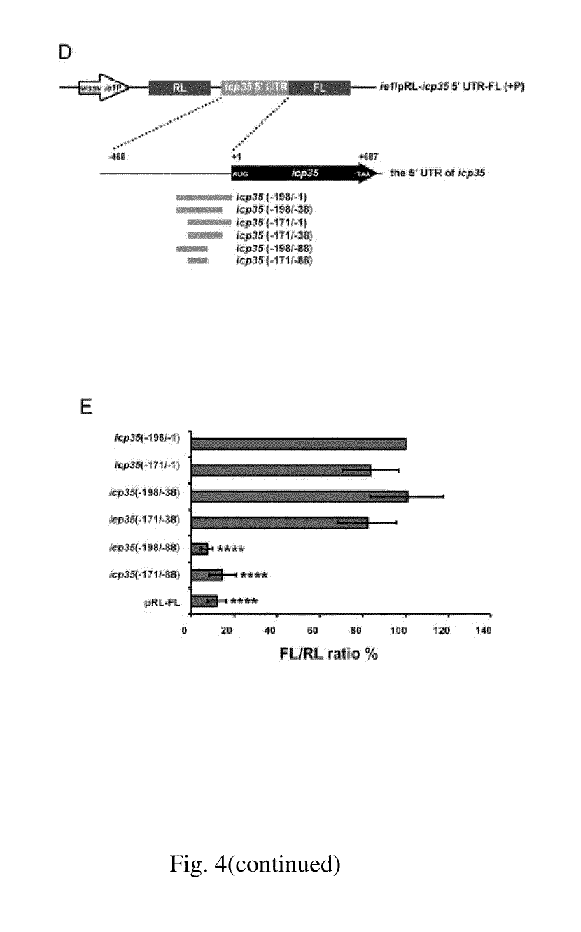

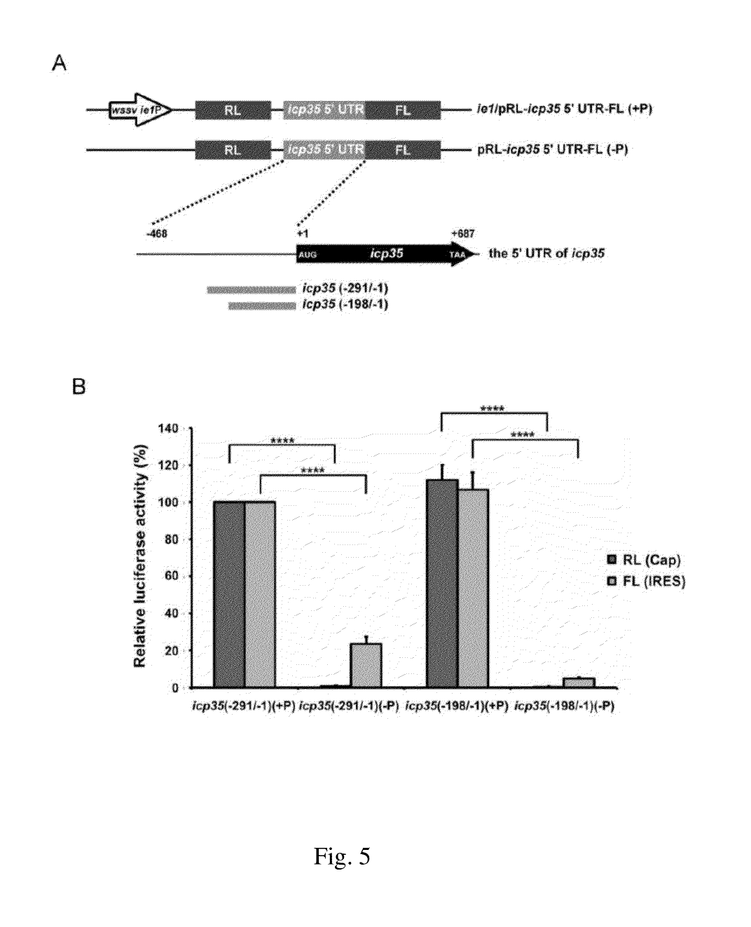

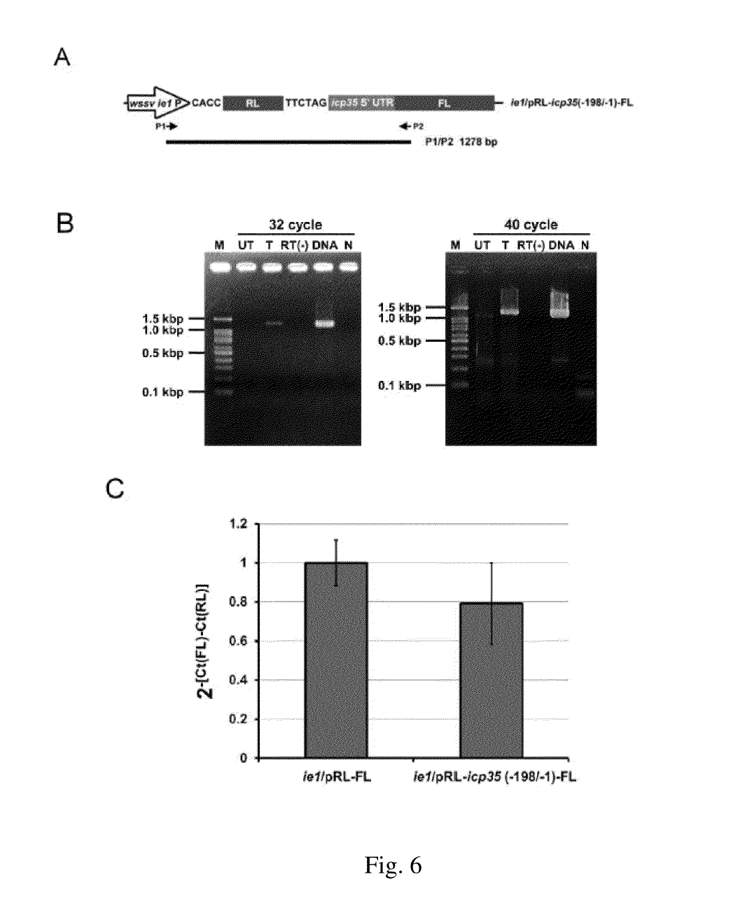

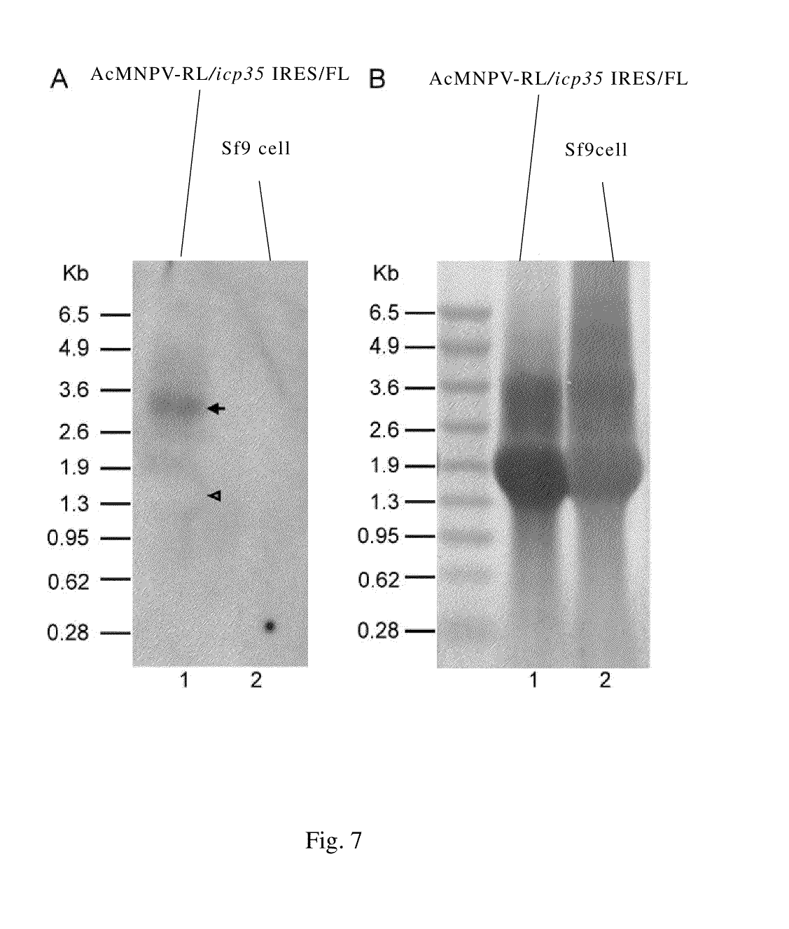

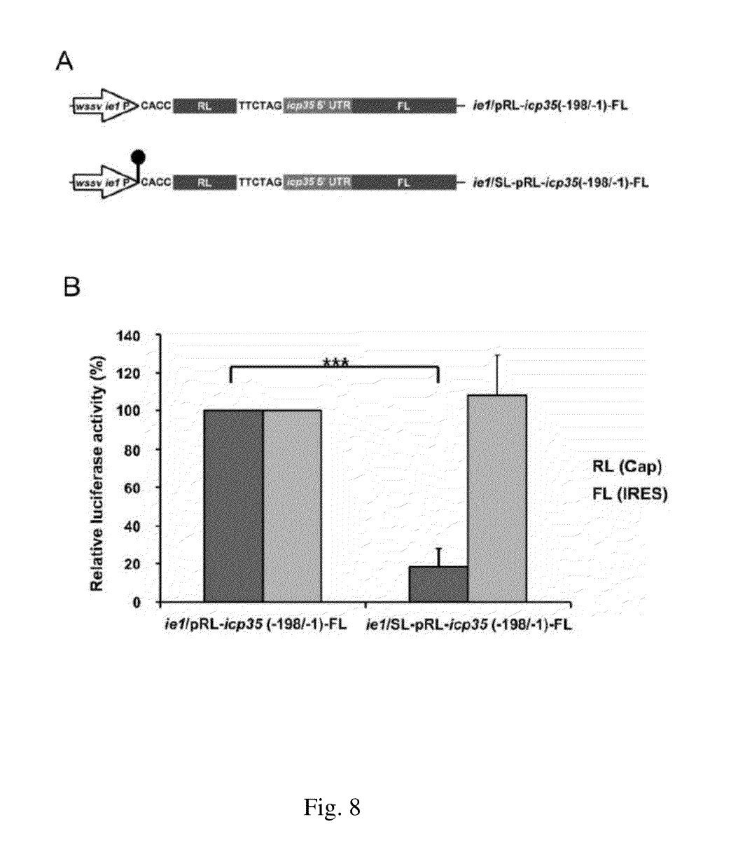

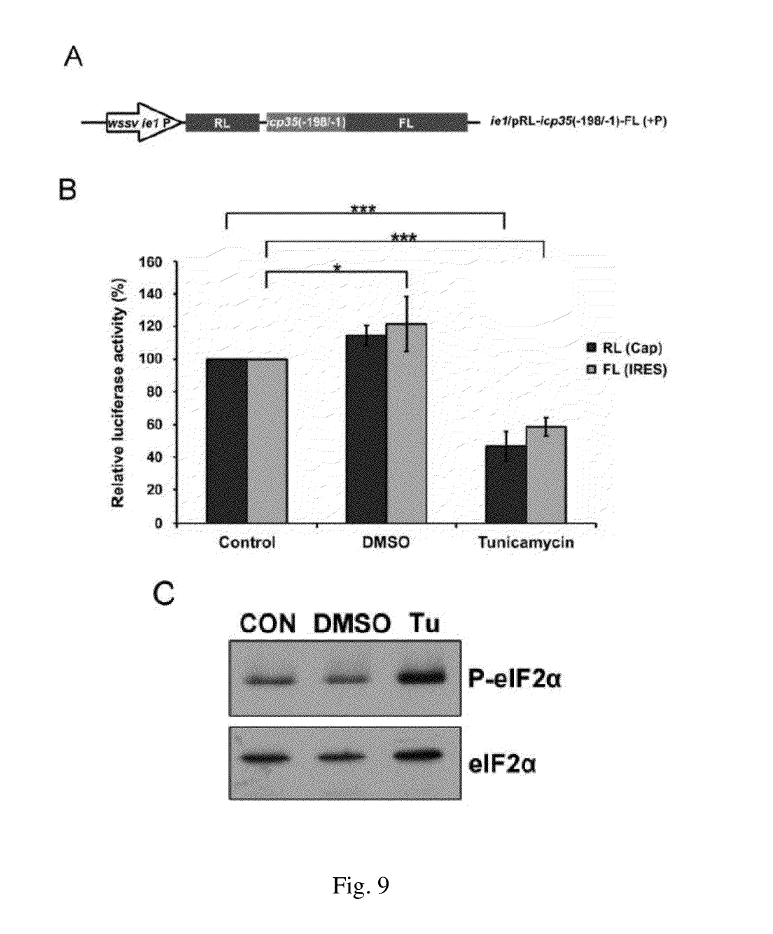

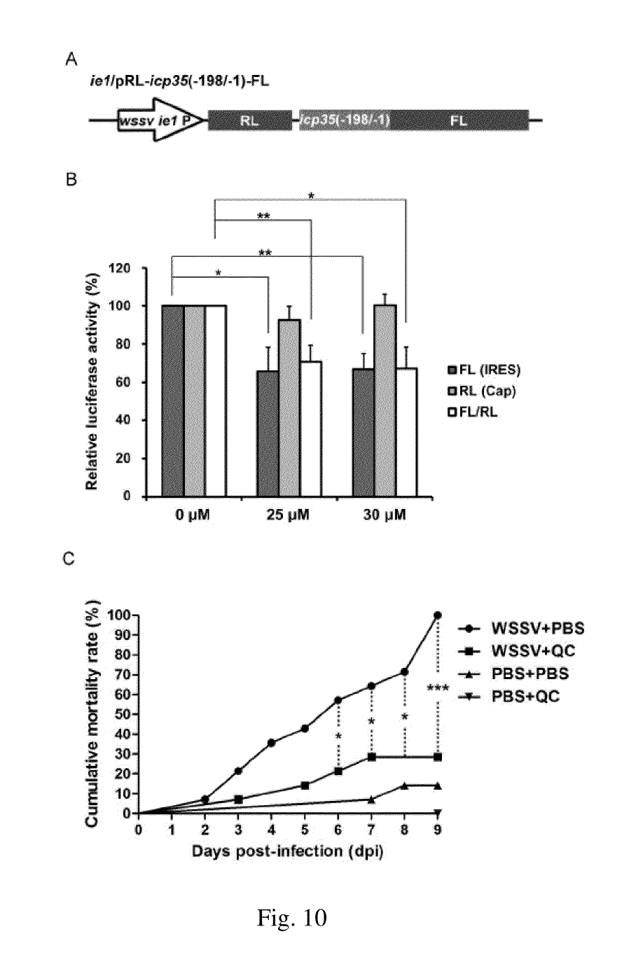

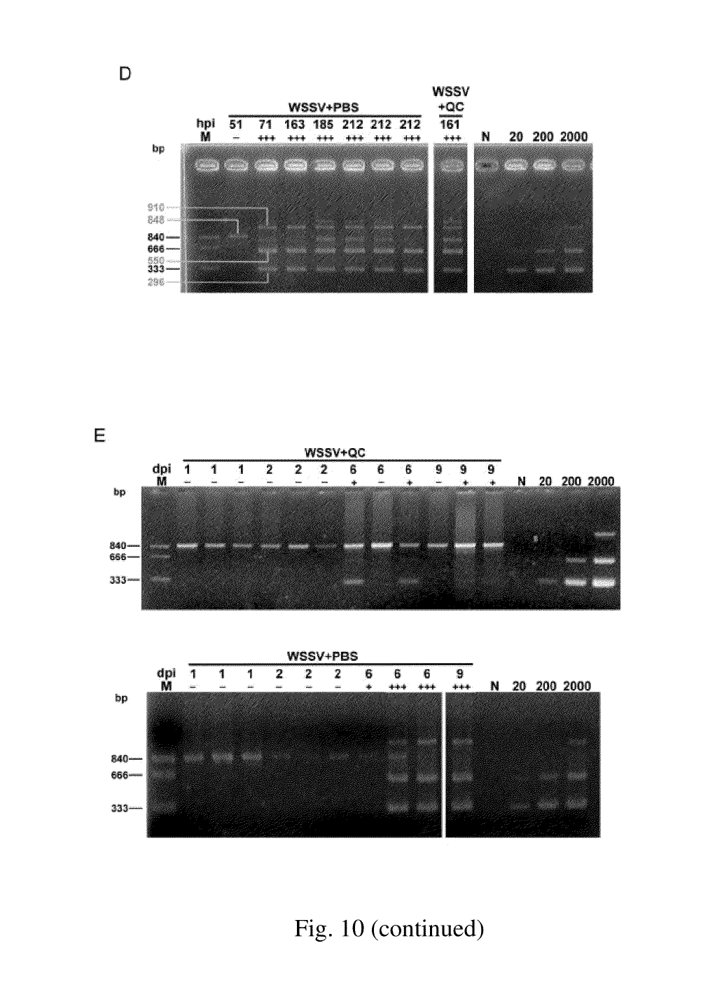

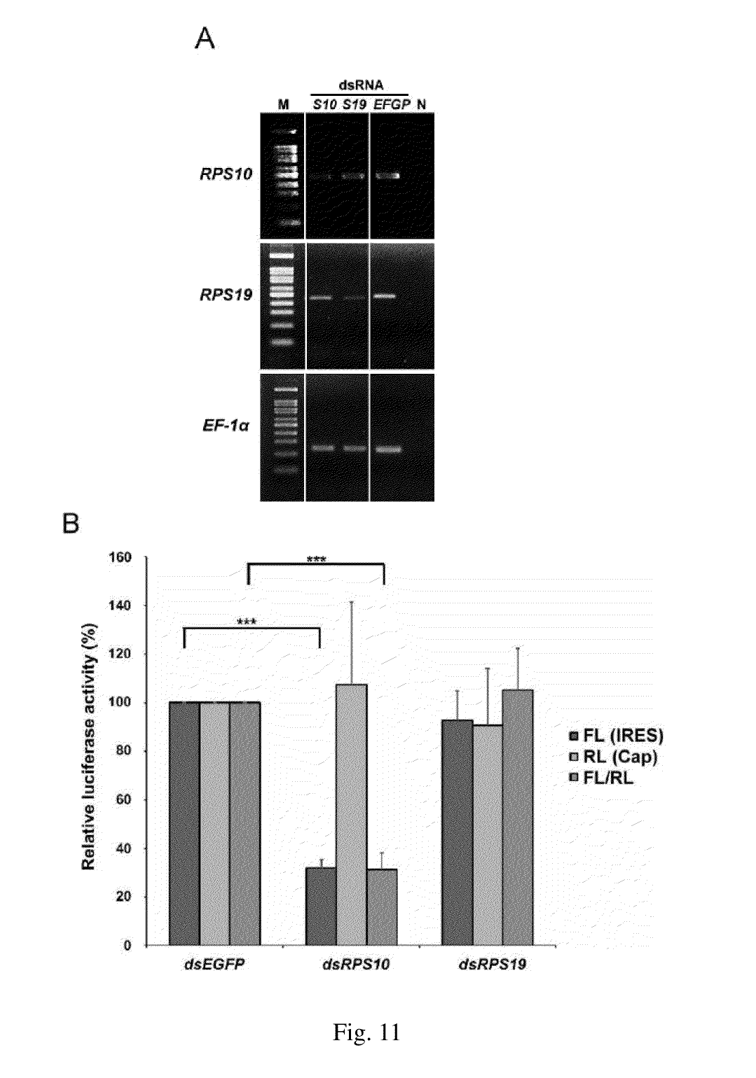

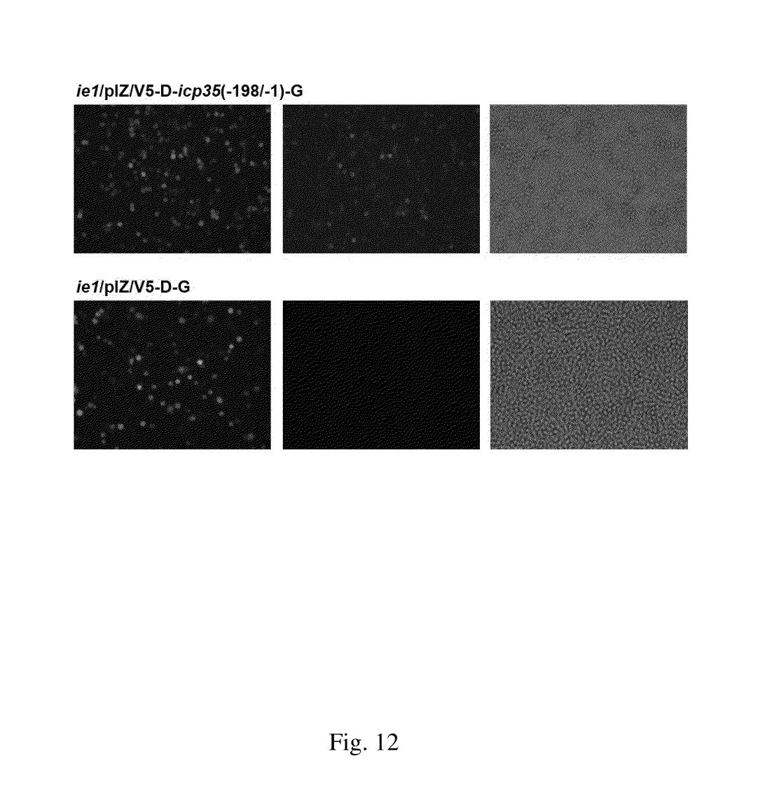

To investigate the importance of ICP35 to WSSV pathogenesis, dsRNA gene silencing was used to knock down the expression of icp35 during WSSV infection. First, RT-PCR analysis was used to confirm the knockdown efficiency of icp35 dsRNA at 24, 48 and 96 hpi in WSSV-challenged shrimp (FIG. 2A), while expression of the immediate early gene ie1 was used to confirm WSSV infection. In the icp35-treated dsRNA group, there was no icp35 or ie1 signal detected in two out three shrimp at 48 hpi. The expression of icp35 and ie1 in the third shrimp was probably due to a combination of incomplete silencing and individual differences. At 96 hpi, in the same group, none of the three sampled shrimp produced an icp35 or ie1 signal.

The expression of the housekeeping gene EF-1α was not affected by icp35 or EGFP dsRNA treatment at 24, 48 or 96 hpi (FIG. 2A). Similar results were found in the mortality study (FIG. 2B). After WSSV challenged, mortality in the EGFP dsRNA and PBS controls very soon reached 100%. By contrast, in the icp35 dsRNA group, two-thirds of the challenged shrimp survived through to the end of the experiment.

FIG. 2C showed that controls of icp35 only or non-challenged EGFP dsRNA had no significant differences as compared with the negative control of PBS only, indicating that the group of icp35 dsRNA or EGFP dsRNA had no effect on shrimp.

Real-time PCR analysis by the IQ REAL WSSV quantitative system (GeneReach Biotechnology Corp.) showed that the WSSV loads of the icp35 and EGFP dsRNA-treated groups were significantly lower than the PBS group (P<0.005) at 48 hpi (FIG. 2D). However, at 96 hpi, WSSV loads remained low only in the icp35 group, while the virus copy number increased markedly in the other groups. Although the very large variation in virus load in the EGFP and PBS groups (SD=5865.49 and 37174.89, respectively) means that this difference did not reach statistical significance with P<0.05, taken together, these results consistently suggest that ICP35 is important for WSSV replication.

3. The 5′ UTR of Icp35 mRNA Mediated Internal Initiation of Translation

To determine if an IRES element was located upstream of icp35, a 468 by region (-468/-1) between multiple repeats TTTTTCTCC and the icp35 translational start codon (ATG, which A is in +1 position) was cloned into the bicistronic vector ie1/pRL-FL with the WSSV ie1(-94/+52) promoter (FIG. 3A). The plasmid was transfected into Sf9 cells, and Renilla (RL) and firefly (FL) luciferase activities were measured 48h after transfection.

In this example, the first cistron (i.e. Renilla) was translated by a cap-dependent translation mechanism, whereas translation of the second cistron (i.e. firefly) would suggest that the intercistronic region contains an IRES element. The previously reported IRES elements associated with VP39B and VP28 were used for comparison, while the empty ie1/pRL-FL bicistronic vector was used as a negative control. These results suggested that the 5′ UTR of icp35 contained an active IRES element (FIG. 3B). Furthermore, the IRES activity of icp35 (i.e. icp35(-468/-1)) appeared to be considerably higher than the two previously reported WSSV IRES elements in vp39b and vp28.

Next step in Examples was to more precisely locate the active IRES element within the icp35 5′ UTR. Based on the predicted RNA secondary structure of the putative IRES region of the 5′ UTR of icp35(-468/-1) (FIG. 4A), various internal sequences containing one or more stem-loops (SL) were selected for insertion into the ie1/pRL-FL bicistronic vector (FIG. 4B), and performed the same dual luciferase assay described above. As shown in FIG. 4C, the shortest IRES fragment that still had a high ratio of FL to RL luciferase activity was (-198/-1). This fragment contained the stem-loops VII, VIII and IX. (The actual ratio of cap-independent to cap-dependent activity for this fragment was 1.32±0.20, but this was adjusted to 100% in the Figure for purposes of comparison.)

Using the same strategy, further refinement of the icp35(-198/-1) sequence showed that the icp35(-171/-38) fragment (FIG. 4D), which contained stem-loops VII and VIII (albeit somewhat modified: Mfold predicted a slightly smaller stem-loop VII and a slightly larger stem-loop VIII), was the smallest that still had a high FL/RL ratio (FIG. 4E). It further appeared that the presence of both of these stem-loops is required for IRES activity because neither icp35(-128/-1) (stem-loops VIII and IX) nor ICP35(-198/-112) (stem-loop VII) were sufficient to produce a high FL/RL ratio on their own (FIG. 4C). It therefore concluded that icp35 IRES activity was supported by stem-loops VII and VIII working together.

4. The icp35 IRES Element Did not Contain a Cryptic Promoter or Splice Sites

To exclude the possibility that stem-loops VII and VIII were harboring a cryptic promoter that was driving the expression of FL, a promoterless assay was done using a bicistronic plasmid with the wssv ie1 promoter removed (FIG. 5A). FIG. 5B shows that while there was still some FL activity induced by the (-291/-1) fragment, only negligible FL activity was induced by the shorter (-198/-1) fragment. Since the shorter fragment contained the entire IRES element (i.e. stem-loops VII and VIII) plus stem-loop IX, it concluded that there was no cryptic promoter in this region.

An alternative explanation of the observed

FL activity of ie1/pRL-icp35(-198/-1)-FL(+P) (FIG. 6A) is that it might be due to an abnormal splicing event. To rule out the possibility that icp35(-198/-1) may contain splice sites, RT-PCR analysis was performed to verify the integrity of the bicistronic transcript in Sf9 cells. Only a single transcript of the expected size (1278 bp) was produced in 32 cycles (FIG. 6B). To rule out the possibility that weak minor bands were not being detected, the same eDNA samples were also subjected to 40 cycles of amplification.

As shown in the oversaturated right-hand panel of FIG. 6B, in addition to the single main transcript, only the same two non-specific minor bands of 250 bp and 220 bp were detected in both the untransfected (UT) and transfected (T) eDNA samples. After confirming the actual size and sequence of the major 1278 by RT-PCR product, we concluded that no abnormal splicing occurred during transient transfection by the bicistronic plasmid. Taken together, it concluded that the FL activity was indeed driven by the bicistronic transcript of ie1/pRL-icp35(-198/-1)-FL through IRES-mediated regulation.

These conclusions were further supported by using quantitative real-time PCR to accurately determine the gene expression levels of RL and FL. As shown in FIG. 6C, the ratio of firefly luciferase to Renilla luciferase

produced by the ie1/pRL-icp35(-198/-1)-FL plasmid was only 80% of that produced by ie1/pRL-FL. A Student's t-test found no significant difference between these two ratios, and this result therefore confirmed that during transfection by the ie1/pRL-icp35(-198/-1)-FL plasmid, no cryptic promoter was acting to increase the mRNA level of FL and no abnormal splicing event was occurring to reduce the mRNA level of RL.

The integrity of the bicistronic mRNA was also confirmed by Northern blot analysis with a DIG-labeled RNA probes for firefly luciferase (FIG. 7). As shown in FIG. 7A, only a 3.2 kb RNA transcript of the expected size was detected. No smaller fragments and no monocistronic firefly luciferase mRNA were observed. It therefore concluded that the firefly luciferase was translated exclusively from the bicistronic mRNA produced by the AcMNPV-RL/icp35 IRES/FL virus.

5. Ribosomal Read-Through Did not Responsible for Icp35 IRES Activity

To rule out the possibility that the FL activity was caused by read-through of the RL termination codon, a stable stem-loop was inserted upstream of the RL ORF (FIG. 8A). The inserted stem-loop significantly reduced the

RL activity of the

ie1/SL-pRL-icp35(-198/-1)-FL plasmid, while the FL activity was not affected (FIG. 8B). It concluded that the observed FL activity was not produced by a ribosomal read-through mechanism.

6. Initiation of Translation on the Icp35 IRES was eIF2-Dependent

To determine whether eIF2 is required for icp35 IRES-mediated translation, the translational machinery was shut down by inducing phosphorylation of eIF2a with the ER stress chemical reagent tunicamycin. It was found that the ER stress induced by tunicamycin inhibited both cap-dependent and icp35 IRES-dependent translation (FIG. 9). These data indicated that active eIF2a was necessary for icp35 IRES activity, and it therefore concluded that initiation of translation on the icp35 IRES was eIF2-dependent.

7. Quinacrine (QC) Reduced Icp35 IRES Activity and has an Anti-WSSV Effect

QC is an intercalating drug that inhibits DNA replication and RNA transcription. Nucleic acid intercalating drugs were shown to inhibit IRES-mediated translation more than cap-dependent translation at low concentrations (10 to 20 μM). It was subsequently shown that QC inhibited the IRES activities of Encephalomyocarditis Virus (EMCV), hepatitis C virus (HCV) and poliovirus in a dose-dependent manner by interacting with the highly complex secondary structures of their IRES regions (see Gasparian, A. V., N. Neznanov, S. Jha, O. Galkin, J. J. Moran, A. V. Gudkov, K. V. Gurova, and A. A. Komar. 2010. Inhibition of encephalomyocarditis virus and poliovirus replication by quinacrine: implications for the design and discovery of novel antiviral drugs. J Virol 84:9390-9397, which was incorporated by reference). The same study also found that the cellular p53 IRES, which has a much less complex secondary structure, is not sensitive to QC. Since the IRES region of icp35 was predicted to be complex (FIG. 4A), it would be expected that transient transfection with the bicistronic plasmid containing the icp35 IRES element (FIG. 10 B) would show reduced FL reporter activity in the presence of QC. As FIG. 10 B showed, QC had no significant effect on the RL value, but at 25 μM and 30 μM, it significantly reduced the FL value and also the FL/RL ratio.

Having shown that QC suppressed icp35 IRES activity in vitro, the effect of this drug on WSSV-challenged shrimp in vivo was next investigated. FIG. 10C showed that after challenge with an inoculum of WSSV, the immediate injection of QC at 5 μg/g significantly reduced the mortality rate. Mortality in the positive control group exceeded 50% at 6 dpi, while over 70% of the WSSV+QC group were still alive at 9 dpi (and in fact continued to survive for more than two weeks; data not shown).

Nested competitive PCR analysis detected a high virus load (heavy infection) in all but one of the shrimp that died (FIG. 10 D). In the replicate groups that were used for sampling by 6 dpi, there was also a heavy infection in two out of three of the surviving shrimp in the WSSV+PBS group (FIG. 10E). At 9 dpi in the WSSV+PBS sample collection group, there was only one surviving shrimp, and this shrimp also had a heavy WSSV infection (FIG. 10E). By contrast, the surviving shrimp in the WSSV+QC sample collection group tested negative or had only a light infection throughout the entire period of the experiment (FIG. 10E).

8. RPS10 Knockdown Selectively Inhibited the Icp35 IRES Activity

Knockdown of certain ribosomal proteins could have a differential effect on cap-dependent versus IRES-mediated

translation. Spodoptera frugierda RPS10 and RPS19 genes were selected for this experiment based on applicant's unpublished proteomics data and a literature review for other viral IRES element (see Babaylova, E., D. Graifer, A. Malygin, J. Stahl, I. Shatsky, and G. Karpova. 2009. Positioning of subdomain IIId and apical loop of domain II of the hepatitis C IRES on the human 40S ribosome. Nucleic Acids Res 37:1141-1151; Otto, G. A., P. J. Lukaysky, A. M. Lancaster, P. Sarnow, and J. D. Puglisi. 2002. Ribosomal proteins mediate the hepatitis C virus IRES-HeLa 40S interaction. RNA 8:913-923; Sarnow, P. 2003. Viral internal ribosome entry site elements: novel ribosome-RNA complexes and roles in viral pathogenesis. J Virol 77:2801-2806. All of them were incorporated by reference). After first using RT-PCR analysis to confirm the knockdown efficiency of RPS10 and RPS19 dsRNA at 72 hpi (FIG. 11A), the results showed that RPS10 knockdown significantly decreased icp35 IRES-mediated FL activity to 40% compared to the EGFP dsRNA control, while the cap-dependent RL activity was not affected (FIG. 11B). RPS19 knockdown had no effect on cap-dependent translation or on IRES-mediated translation (FIG. 11B). These data indicated that RPS10 is required for icp35 IRES activity.

B. Preparation and Analysis for the Dual-Fluorescence Reporter Vector (Ie1/pIZV5- DR-Icp 35 IRES-GFP, Including a Sequence of SEQ ID NO: 3)

### Example 1

### Preparation of Ie1/pIZV5- DR-Icp 35 IRES-GFP

The pIZ/V5-His PCR fragment without the OpIE2 promoter (base 4-552) was amplified using KOD⁺ Taq polymerase (TOYOBO) with the primer set pIZ/V5-His-HindIII-F-ΔOpIE2p and pIZ/V5-His-R-ΔOpIE2p (Table 1) from the pIZ/V5-His vector. The WSSV ie1 promoter was amplified using KOD+Taq polymerase (TOYOBO) with the primer set ie1-Pro2-Bgl II and ie1-R-HindIII (Table 1), digested with HindIII and then cloned into the HindIII site of pIZ/V5-HisAOpIE2 (4-552) to generate the construct ie1/pIZ/V5-His. The pBacDIRE plasmid was a gift from Dr. Wu (Chung Yuan Christian University, Taiwan, ROC)(Chen et al., 2005, which was incorporated by reference). The fragment DIRE containing DsRed, IRES, and EGFP gene was amplified using the primer set DsRed-KpnI-F and EGFP-R from the pBacDIRE plasmid (Table 1). The PCR amplified fragment was digested with KpnI, and then cloned into KpnI-XbaI (Klenow filled) sites of ie1/pIZ/V5-His vector to generate the construct ie1/pIZ/V5-DIRE. The IRES was removed by digestion with BamHI from the ie1/pIZ/V5-DIRE, and then religated to produce the construct ie1/pIZ/V5-D-E. The icp35(-198/-1) IRES element was amplified with the primer set icp35(-198)-BamHI-F and icp35(-1)-BamHI-R (Table 1) and then cloned into the BamHI site of the ie1/pIZ/V5-D-E to produce the bicistronic plasmid ie1/pIZ/V5-D-icp35(-198/-1)-E.

Results

FIG. 12 illustrated the result for the expression of a bicistronic plasmid containing the icp35 IRES element, ie1/pIZ-V5-DR-icp35 IRES-EGFP, which was transfected into Sf9 cells using Effectene transfection reagent (Qiagen). At 72 post-transfection, both of the expression of DsRed and EGFP was observed under fluorescence microscope (Olympus) and photographed. An empty vector without any IRES element, ie1/pIZ-V5-DR-EGFP, was used as a negative control. To further confirm whether the icp35(-198/-1) IRES could be applied for expression of other reporter genes, we construct a dual-fluorescence reporter plasmid. The first cistron DsRed of this plasmid is expressed by a cap-dependent translation, and the second cistron EGFP is expressed via the icp35(-198/-1) IRES-mediated translation. The icp35(-198/-1) IRES containing bicistronic plasmid was transfected into Sf9 cells. At 72 h post-transfection, both of the DsRed and EGFP signals were observed using the fluorescent microscopy (Olympus). Therefore, we confirm that icp35(-198/-1) IRES not only could be applied for expression of luminescent protein, but also for expression of fluorescent protein. Taken together, the icp35(-198/-1) IRES element can successfully regulate the translation of at least two reporter genes. In addition, the IRES has a high IRES activity, so it is valuable for development of multi-gene expression vectors.

Embodiments of the inventive concept of the present invention may be modified in various forms, and the scope and spirit of the present invention should not be construed as being limited by the above-described embodiments. Therefore, the above-disclosed Embodiments are to be considered illustrative, and not restrictive, and the appended claims are intended to cover all such modifications, enhancements, and other embodiments, which fall within the true spirit and scope of the present invention.

TABLE 1
Primers used in all Examples

| SEQ ID | | | |
|---|---|---|---|
| NO: | Primer name | Sequence | Usage |
| 5 | ie1 promoter-SacI-F | 5′-AGGAGCTCCCTTGTTACTCATTTATTCCTA-3′ | Plasmid construction for |
| | | | dual luciferase assay |
| 6 | ie1 promoter-NheI-R | 5′-CCGCTAGCCTTGAGTGGAGAGAGAGA-3′ | Plasmid construction for |
| | | | dual luciferase assay |
| 7 | icp35(-468)-F | 5′-ATGTTACATTCTTTATATAATGGTGAATC-3′ | Plasmid construction for |
| | | | dual luciferase assay |
| 8 | icp35(-1)-NcoI-R | 5′-TTTCCATGGTTTGGGGGTTATTTTTGGA-3′ | Plasmid construction for |
| | | | dual luciferase assay |
| 9 | icp35(-426)-SmaI-F | 5′-GGCCCGGGATGGTTTTTGTCTTTTTTAAAG-3′ | Plasmid construction for |
| | | | dual luciferase assay |
| 10 | icp35(-384)-F | 5′-AAGCCTTTTTATATTTATTGAAGATAA-3′ | Plasmid construction for |
| | | | dual luciferase assay |
| 11 | icp35(-344)-SmaI-F | 5′-AACCCGGGAATAATCATGTTAATAACAC-3′ | Plasmid construction for |
| | | | dual luciferase assay |
| 12 | icp35(-291)-SmaI-F | 5′-GGCCCGGGTTATCAAACACTATGCATTTC-3′ | Plasmid construction for |
| | | | dual luciferase assay |
| 13 | icp35(-198)-SmaI-F | 5′-TTCCCGGGGTTTCTGGCACATATAGTGATG-3′ | Plasmid construction for |
| | | | dual luciferase assay |
| 14 | icp35(-128)-SmaI-F | 5′-TTCCCGGGTGCCACGAGTGTATATATAGGA-3′ | Plasmid construction for |
| | | | dual luciferase assay |
| 15 | icp35(-171)-SmaI-F | 5′-TTCCCCGGGAGTTGGCAACTCTATCACTA-3′ | Plasmid construction for |
| | | | dual luciferase assay |
| 16 | icp35(-112)-NcoI-R | 5′-TTTCCATGGATATACACTCGTGGCACGGTG-3′ | Plasmid construction for |
| | | | dual luciferase assay |
| 17 | icp35(-1)-F | 5′-TTTGGGGGTTATTTTTGGAACTCGTG-3′ | Plasmid construction for |
| | | | dual luciferase assay |
| 18 | icp35(-468)-NcoI-R | 5′-TTTCCATGGTGTTACATTCTTTATATAATG-3′ | Plasmid construction for |
| | | | dual luciferase assay |
| 19 | icp35(-426)-NcoI-R | 5′-GGCCATGGATGGTTTTTGTCTTTTTTAAAG-3′ | Plasmid construction for |
| | | | dual luciferase assay |
| 20 | icp35(-88)-NcoI-R | 5′-TTTCCATGGGTGGTGCAGGATCGGGGGTC-3′ | Plasmid construction for |
| | | | dual luciferase assay |
| 21 | icp35(-38)-NcoI-R | 5′-TTTCCATGGAGTAGTAGTATTAGGTTAG-3′ | Plasmid construction for |
| | | | dual luciferase assay |
| 22 | vp31-IRF1-N123-SmaI | 5′-CACCCGGGCGAATTGTTGAAGAACACTG-3′ | Plasmid construction for |
| | | | dual luciferase assay |
| 23 | vp39b-IRR1-C919-NcoI | 5′-AACCATGGCTAAGCGATACTTTAATTG-3′ | Plasmid construction for |
| | | | dual luciferase assay |
| 24 | vp28-IRES-SmaI-F | 5′-TGCCCGGGTAGACCCTGGCTTACTGTA-3′ | Plasmid construction for |
| | | | dual luciferase assay |
| 25 | vp28-IRES-NcoI-R | 5′-TCCCATGGGACGAGTTTTTTTCTTTATC-3′ | Plasmid construction for |
| | | | dual luciferase assay |
| 26 | P1(ie1P[+3/+31]-F) | 5′-CACAAGAGCGCACACACACGCTTATAACT-3′ | RT-PCR assay |
| 27 | P2(pRL-FL-R1241) | 5′-CCAGCGGTTCCATCTTCCAGCGGATA-3′ | RT-PCR assay |
| 28 | icp35-F1 | 5′-ATGGTCTCTTCTAGAACATCAACA-3′ | For dsRNA-mediated gene silencing |
| | | | and RT-PCR assay |
| 29 | icp35-R687 | 5′-TTACCAACAAGGATCATCAATCA-3′ | For dsRNA-mediated gene silencing |
| | | | and RT-PCR assay |
| 30 | icp35-dsRNA-T7-F1 | 5′-TAATACGACTCACTATAGGGAGAATGGTC | For dsRNA-mediated gene silencing |
| | | TCTTCTAGAACATCAACA-3′ | |
| 31 | icp35-dsRNA-T7-R687 | 5′-TAATACGACTCACTATAGGGAGATTACCA | For dsRNA-mediated gene silencing |
| | | ACAAGGATCATCAATCA-3′ | |
| 32 | EGFP-F | 5′-GTTCAGCGTGTCCGGCGAG-3′ | For dsRNA-mediated gene silencing |
| 33 | EGFP-R | 5′-GTTCTTCTGCTTGTCGGCC-3′ | For dsRNA-mediated gene silencing |
| 34 | vp28-IRES-NcoI-R | 5′-TCCCATGGGACGAGTTTTTTTCTTTATC-3′ | Plasmid construction for |
| | | | dual luciferase assay |
| 35 | P1(ie1P[+3/+31]-F) | 5′-CACAAGAGCGCACACACACGCTTATAACT-3′ | RT-PCR assay |
| 36 | P2(pRL-FL-R1241) | 5′-CCAGCGGTTCCATCTTCCAGCGGATA-3′ | RT-PCR assay |
| 37 | icp35-F1 | 5′-ATGGTCTCTTCTAGAACATCAACA-3′ | For dsRNA-mediated gene silencing |
| | | | and RT-PCR assay |
| 38 | EGFP-F | 5′-GTTCAGCGTGTCCGGCGAG-3′ | For dsRNA-mediated gene silencing |
| 39 | EGFP-R | 5′-GTTCTTCTGCTTGTCGGCC-3′ | For dsRNA-mediated gene silencing |
| 40 | EGFP-T7-F | 5′-TAATACGACTCACTATAGGGAGAGTTCAG | For dsRNA-mediated gene silencing |
| | | CGTGTCCGGCGAG-3′ | |
| 41 | EGFP-T7-R | 5′-TAATACGACTCACTATAGGGAGAGTTCTT | For dsRNA-mediated gene silencing |
| | | CTGCTTGTCGGCC-3′ | |
| 42 | ie1-F | 5′-GACTCTACAAATCTCTTTGCCA-3′ | RT-PCR assay |
| 43 | ie1-R | 5′-CTACCTTTGCACCAATTGCTAG-3′ | RT-PCR assay |
| 44 | EF-1α-F | 5′-GGAGATGCACCACGAAGCTC-3′ | RT-PCR assay |
| 45 | EF-1α-R | 5′-TTGGGTCCGGCTTCCAGTTC-3′ | RT-PCR assay |
| 46 | Rluc-qPCR-F | 5′-AACGCGGCCTCTTCTTATTT-3′ | Quantitative RT-PCR assay |
| 47 | Rluc-qPCR-R | 5′-ATTTGCCTGATTTGCCCATA-3′ | Quantitative RT-PCR assay |
| 48 | Fluc-qPCR-F | 5′-GAGGTTCCATCTGCCAGGTA-3′ | Quantitative RT-PCR assay |
| 49 | Fluc-qPCR-R | 5′-CCGGTATCCAGATCCACAAC-3′ | Quantitative RT-PCR assay |
| 50 | Sf9-EF-1α-qPCR-F | 5′-TGATCTACAAATGCGGTGGT-3′ | Quantitative RT-PCR assay |
| 51 | Sf9-EF-1α-qPCR-R | 5′-CTCAGCCTTCAGTTTGTCCA-3′ | Quantitative RT-PCR assay |
| 52 | FL-F | 5′-GGAACCGCTGGAGAGCAACT-3′ | Northern blot analysis |
| 53 | FL-R | 5′-CGAAGGACTCTGGCACAAAATCGT-3′ | Northern blot analysis |
| 54 | icp35(-198)-BamHI | 5′-TTTGGATCCGTTTCTGGCACATATAGTGATG-3′ | Plasmid construction for |
| | | | recombinant virus |
| 55 | icp35(-1)-XhoI | 5′-TTTCTCGAGTTTGGGGGTTATTTTTGGA-3′ | Plasmid construction for |
| | | | recombinant virus |
| 56 | Sf9-RPS10-F | 5′-TGTTGATGCCTAAACAGAATCGCGT-3′ | For dsRNA-mediated gene silencing |
| | | | and RT-PCR assay |
| 57 | Sf9-RPS10-R | 5′-TTAAGGTGCAGGCCTGCCTCGTCCGA-3′ | For dsRNA-mediated gene silencing |
| | | | and RT-PCR assay |
| 58 | Sf9-RPS10-dsRNA-T7F | 5′-TAATACGACTCACTATAGGGAGATGTTGATGC-3′ | For dsRNA-mediated gene silencing |
| 59 | Sf9-RPS19-dsRNA-T7R | 5′-TAATACGACTCACTATAGGGAGATTACAGAAC-3′ | For dsRNA-mediated gene silencing |
| 60 | pIZ/V5-His-HindIII-F- | 5′-CTGTTCGAATTTAAAGCTTGGTACCGAG-3′ | Construction for ie1/pIZ/V5 |
| | ΔOpIE2p | | |
| 61 | pIZ/V5-His-R-ΔOpIE2p | 5′-ATCCAGACATGATAAGATACATTGATGAG-3′ | Construction for ie1/pIZ/V5 |
| 62 | ie1-Pro2-Bg1 II | 5′-GGAAGATCTGATGATGGTGATGTTTCTAGG-3′ | Construction for ie1/pIZ/V5 |
| 63 | ie1-R-HindIII | 5′-GAAAAGCTTCTTGAGTGGAGAGAGAG-3′ | Construction for ie1/pIZ/V5 |
| 64 | DsRed-KpnI-F | 5′-AAAGGTACCATGGTGCGCTCCTCCAAG-3′ | Construction for ie1/pIZ/V5-DIRE |
| 65 | EGFP-R | 5′-TTACTTGTACAGCTCGTCCATGCCGAG-3′ | Construction for ie1/pIZ/V5-D-IR-E |
| 66 | icp35(-198)-BamHI-F | 5′-CGCGGATCCGTTTCTGGCACATATAGTGATG-3′ | Construction for |
| | | | ie1/pIZ/V5-D-icp35(-198/-1)-E |
| 67 | icp35(-1)-BamHI-R | 5′-AAGGATCCTTTGGGGGTTATTTTTGGA-3′ | Construction for |
| | | | ie1/pIZ/V5-D-icp35(-198/-1)-E |

What is claimed is:

1. An expression vector comprising an internal ribosome entry site (IRES) element, which comprises a sequence of SEQ ID NO: 1.

2. The expression vector of claim 1, wherein the sequence is an IRES element of a gene icp35 in White spot syndrome virus (WSSV).

3. The expression vector of claim 1, wherein the expression vector is a dual-gene expression vector.

4. The expression vector of claim 3, wherein the dual-gene expression vector is a dual-luciferase reporter vector.

5. The expression vector of claim 4, wherein the dual-luciferase reporter vector comprises a sequence of SEQ ID NO: 2.

6. The expression vector of claim 3, wherein the dual-gene expression vector is a dual-fluorescence reporter vector.

7. The expression vector of claim 6, wherein the dual-fluorescence reporter vector comprises a sequence of SEQ ID NO: 3.

8. A multigene expression system, which comprises the expression vector according to claim 1.

9. The multigene expression system of claim 8, which is an insect cell expression system or a Crustacean cell expression system.

10. The multigene expression system of claim 9, which is a Spodoptera frugiperda (Sf9) expression system.

11. The multigene expression system of claim 8, wherein the expression vector comprises an IRES element of a gene icp35 in White spot syndrome virus (WSSV).

12. The multigene expression system of claim 8, wherein the expression vector is a dual-gene expression vector.

13. The multigene expression system of claim 12, wherein the dual-gene expression vector is a dual-luciferase reporter vector.

14. The multigene expression system of claim 13, wherein the dual-luciferase reporter vector comprises a sequence of SEQ ID NO: 2.

15. The multigene expression system of claim 12, wherein the dual-gene expression vector is a dual-fluorescence reporter vector.

16. The multigene expression system of claim 15, wherein the dual-fluorescence reporter vector comprises a sequence of SEQ ID NO: 3.


SEQUENCE LISTING (67 sequences)

SEQ ID NO: 1 - DNA, 198 bp, Artificial Sequence
  internal ribosome entry site (IRES)
gtttctggcacatatagtgatggtacgagttggcaactctatcactaccccctctcccccccccccaccgtgccacgagtgtatatataggacccccgatcctgcaccacagcacacttgatcatcgtaatcatcagcatcctaacctaatactactactacttgttctacccacgagttccaaaaataacccccaaa

SEQ ID NO: 2 - DNA, 2963 bp, Artificial Sequence
  Artificial Sequence
gagctcccttgttactcatttattcctagaaatggtgtaatcgctgttgtgggcggagcatatttgtgtatataagagcccgtgttagctcctcgattcagtcacaagagcgcacacacacgcttataactagctctctctctccactcaaggctagccaccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataattctagcccggggtttctggcacatatagtgatggtacgagttggcaactctatcactaccccctctcccccccccccaccgtgccacgagtgtatatataggacccccgatcctgcaccacagcacacttgatcatcgtaatcatcagcatcctaacctaatactactactacttgttctacccacgagttccaaaaataacccccaaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 3 - DNA, 1634 bp, Artificial Sequence
  sequence for plasmid synthesis
atggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaagactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagcgaattcatgggatccgtttctggcacatatagtgatggtacgagttggcaactctatcactaccccctctcccccccccccaccgtgccacgagtgtatatataggacccccgatcctgcaccacagcacacttgatcatcgtaatcatcagcatcctaacctaatactactactacttgttctacccacgagttccaaaaataacccccaaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 4 - DNA, 60 bp, Artificial Sequence
  polynucleotide synthesis
gctagcggtacggcagtgccgtacgacgaattcgtcgtacggcactgccgtaccgctagc

SEQ ID NO: 5 - DNA, 30 bp, Artificial Sequence
  primer set
aggagctcccttgttactcatttattccta

SEQ ID NO: 6 - DNA, 26 bp, Artificial Sequence
  primer set
ccgctagccttgagtggagagagaga

SEQ ID NO: 7 - DNA, 29 bp, Artificial Sequence
  primer set
atgttacattctttatataatggtgaatc

SEQ ID NO: 8 - DNA, 28 bp, Artificial Sequence
  primer set
tttccatggtttgggggttatttttgga

SEQ ID NO: 9 - DNA, 30 bp, Artificial Sequence
  primer set
ggcccgggatggtttttgtcttttttaaag

SEQ ID NO: 10 - DNA, 27 bp, Artificial Sequence
  primer set
aagcctttttatatttattgaagataa

SEQ ID NO: 11 - DNA, 28 bp, Artificial Sequence
  primer set
aacccgggaataatcatgttaataacac

SEQ ID NO: 12 - DNA, 29 bp, Artificial Sequence
  primer set
ggcccgggttatcaaacactatgcatttc

SEQ ID NO: 13 - DNA, 30 bp, Artificial Sequence
  primer set
ttcccggggtttctggcacatatagtgatg

SEQ ID NO: 14 - DNA, 30 bp, Artificial Sequence
  primer set
ttcccgggtgccacgagtgtatatatagga

SEQ ID NO: 15 - DNA, 29 bp, Artificial Sequence
  primer set
ttccccgggagttggcaactctatcacta

SEQ ID NO: 16 - DNA, 30 bp, Artificial Sequence
  primer set
tttccatggatatacactcgtggcacggtg

SEQ ID NO: 17 - DNA, 26 bp, Artificial Sequence
  primer set
tttgggggttatttttggaactcgtg

SEQ ID NO: 18 - DNA, 30 bp, Artificial Sequence
  primer set
tttccatggtgttacattctttatataatg

SEQ ID NO: 19 - DNA, 30 bp, Artificial Sequence
  primer set
ggccatggatggtttttgtcttttttaaag

SEQ ID NO: 20 - DNA, 29 bp, Artificial Sequence
  primer set
tttccatgggtggtgcaggatcgggggtc

SEQ ID NO: 21 - DNA, 28 bp, Artificial Sequence
  primer set
tttccatggagtagtagtattaggttag

SEQ ID NO: 22 - DNA, 28 bp, Artificial Sequence
  primer set
cacccgggcgaattgttgaagaacactg

SEQ ID NO: 23 - DNA, 27 bp, Artificial Sequence
  primer set
aaccatggctaagcgatactttaattg

SEQ ID NO: 24 - DNA, 27 bp, Artificial Sequence
  primer set
tgcccgggtagaccctggcttactgta

SEQ ID NO: 25 - DNA, 28 bp, Artificial Sequence
  primer set
tcccatgggacgagtttttttctttatc

SEQ ID NO: 26 - DNA, 29 bp, Artificial Sequence
  primer set
cacaagagcgcacacacacgcttataact

SEQ ID NO: 27 - DNA, 26 bp, Artificial Sequence
  primer set
ccagcggttccatcttccagcggata

SEQ ID NO: 28 - DNA, 24 bp, Artificial Sequence
  primer set
atggtctcttctagaacatcaaca

SEQ ID NO: 29 - DNA, 23 bp, Artificial Sequence
  primer set
ttaccaacaaggatcatcaatca

SEQ ID NO: 30 - DNA, 47 bp, Artificial Sequence
  primer set
taatacgactcactatagggagaatggtctcttctagaacatcaaca

SEQ ID NO: 31 - DNA, 46 bp, Artificial Sequence
  primer set
taatacgactcactatagggagattaccaacaaggatcatcaatca

SEQ ID NO: 32 - DNA, 19 bp, Artificial Sequence
  primer set
gttcagcgtgtccggcgag

SEQ ID NO: 33 - DNA, 19 bp, Artificial Sequence
  primer set
gttcttctgcttgtcggcc

SEQ ID NO: 34 - DNA, 28 bp, Artificial Sequence
  primer set
tcccatgggacgagtttttttctttatc

SEQ ID NO: 35 - DNA, 29 bp, Artificial Sequence
  primer set
cacaagagcgcacacacacgcttataact

SEQ ID NO: 36 - DNA, 26 bp, Artificial Sequence
  primer set
ccagcggttccatcttccagcggata

SEQ ID NO: 37 - DNA, 24 bp, Artificial Sequence
  primer set
atggtctcttctagaacatcaaca

SEQ ID NO: 38 - DNA, 19 bp, Artificial Sequence
  primer set
gttcagcgtgtccggcgag

SEQ ID NO: 39 - DNA, 19 bp, Artificial Sequence
  primer set
gttcttctgcttgtcggcc

SEQ ID NO: 40 - DNA, 42 bp, Artificial Sequence
  primer set
taatacgactcactatagggagagttcagcgtgtccggcgag

SEQ ID NO: 41 - DNA, 42 bp, Artificial Sequence
  primer set
taatacgactcactatagggagagttcttctgcttgtcggcc

SEQ ID NO: 42 - DNA, 22 bp, Artificial Sequence
  primer set
gactctacaaatctctttgcca

SEQ ID NO: 43 - DNA, 22 bp, Artificial Sequence
  primer set
ctacctttgcaccaattgctag

SEQ ID NO: 44 - DNA, 20 bp, Artificial Sequence
  primer set
ggagatgcaccacgaagctc

SEQ ID NO: 45 - DNA, 20 bp, Artificial Sequence
  primer set
ttgggtccggcttccagttc

SEQ ID NO: 46 - DNA, 20 bp, Artificial Sequence
  primer set
aacgcggcctcttcttattt

SEQ ID NO: 47 - DNA, 20 bp, Artificial Sequence
  primer set
atttgcctgatttgcccata

SEQ ID NO: 48 - DNA, 20 bp, Artificial Sequence
  primer set
gaggttccatctgccaggta

SEQ ID NO: 49 - DNA, 20 bp, Artificial Sequence
  primer set
ccggtatccagatccacaac

SEQ ID NO: 50 - DNA, 20 bp, Artificial Sequence
  primer set
tgatctacaaatgcggtggt

SEQ ID NO: 51 - DNA, 20 bp, Artificial Sequence
  primer set
ctcagccttcagtttgtcca

SEQ ID NO: 52 - DNA, 20 bp, Artificial Sequence
  primer set
ggaaccgctggagagcaact

SEQ ID NO: 53 - DNA, 24 bp, Artificial Sequence
  primer set
cgaaggactctggcacaaaatcgt

SEQ ID NO: 54 - DNA, 31 bp, Artificial Sequence
  primer set
tttggatccgtttctggcacatatagtgatg

SEQ ID NO: 55 - DNA, 28 bp, Artificial Sequence
  primer set
tttctcgagtttgggggttatttttgga

SEQ ID NO: 56 - DNA, 25 bp, Artificial Sequence
  primer set
tgttgatgcctaaacagaatcgcgt

SEQ ID NO: 57 - DNA, 26 bp, Artificial Sequence
  primer set
ttaaggtgcaggcctgcctcgtccga

SEQ ID NO: 58 - DNA, 32 bp, Artificial Sequence
  primer set
taatacgactcactatagggagatgttgatgc

SEQ ID NO: 59 - DNA, 32 bp, Artificial Sequence
  primer set
taatacgactcactatagggagattacagaac

SEQ ID NO: 60 - DNA, 28 bp, Artificial Sequence
  primer set
ctgttcgaatttaaagcttggtaccgag

SEQ ID NO: 61 - DNA, 29 bp, Artificial Sequence
  primer set
atccagacatgataagatacattgatgag

SEQ ID NO: 62 - DNA, 30 bp, Artificial Sequence
  primer set
ggaagatctgatgatggtgatgtttctagg

SEQ ID NO: 63 - DNA, 26 bp, Artificial Sequence
  primer set
gaaaagcttcttgagtggagagagag

SEQ ID NO: 64 - DNA, 27 bp, Artificial Sequence
  primer set
aaaggtaccatggtgcgctcctccaag

SEQ ID NO: 65 - DNA, 27 bp, Artificial Sequence
  primer set
ttacttgtacagctcgtccatgccgag

SEQ ID NO: 66 - DNA, 31 bp, Artificial Sequence
  primer set
cgcggatccgtttctggcacatatagtgatg

SEQ ID NO: 67 - DNA, 27 bp, Artificial Sequence
  primer set
aaggatcctttgggggttatttttgga